(19) United States
(12) Patent Application Publication
(10) Pub. No.: US 2025/0352634 A1
(43) Pub. Date: Nov. 20, 2025

(54) Coxsackievirus B Compositions and Methods Of Use Thereof

(71) Applicant: Vactech OY, Tampere (FI)

(72) Inventors: Heikki Hyöty, Tampere (FL); Mikael Knip, Tampere (FL); Francisco Leon, Red Bank, NJ (US)

(21) Appl. No.: 18/703,256

(22) PCT Filed: Oct. 24, 2022
(86) PCT No.: PCT/US2022/078581

§ 371 Date: Apr. 19, 2024

Related U.S. Application Data
(60) Provisional application No. 63/271,546, filed on Oct. 25, 2021.
(60) Provisional application No. 63/273,723, filed on Oct. 29, 2021.
(60) Provisional application No. 63/321,911, filed on Mar. 21, 2022.

(51) Int. Cl. A61K 39/125; A61K 9/00; A61K 39/00; A61P 31/14; C12N 7/00

(52) U.S. Cl. CPC A61K 39/125; A61K 9/0019; A61P 31/14; C12N 7/00; A61K 2039/5252; A61K 2039/5258; A61K 2039/54; A61K 2039/545; A61K 2039/55; A61K 2039/575; A61K 2039/70; C12N 2770/32323; C12N 2770/32334; C12N 2770/32363

(57) ABSTRACT

The present disclosure provides coxsackievirus B (CVB) compositions and methods of use of such compositions to induce an immune response to CVB in an individual.

### CROSS-REFERENCE

This application claims the benefit of PCT Patent Application No. PCT/US2022/078581, filed Oct. 24, 2022, which application claims the benefit of U.S. Provisional Patent Application No. 63/271,546, filed Oct. 25, 2021, U.S. Provisional Patent Application No. 63/273,723, filed, Oct. 29, 2021, and U.S. Provisional Patent Application No. 63/321,911, filed, Mar. 21, 2022, which applications are incorporated herein by reference in their entirety.

### INCORPORATION-BY-REFERENCE OF SEQUENCE LISTING PROVIDED AS A SEQUENCE LISTING XML FILE

A Sequence Listing is provided herewith as a Sequence Listing XML, “PRVN-V003_SEQ_LIST” created on Jul. 7, 2025 and having a size of 4,169 bytes. The contents of the Sequence Listing XML are incorporated by reference herein in their entirety.

### INTRODUCTION

Coxsackievirus Group B (CVB) is a member of the family Picornaviridae, genus Enterovirus. Six serotypes (1-6) of CVB are recognized: CVB1, CVB2, CVB3, CVB4, CVB5, and CVB6. CVB can cause a variety of diseases, including gastrointestinal illness, myocarditis, pneumonia, aseptic meningitis, encephalitis, and hepatitis.

### SUMMARY

The present disclosure provides coxsackievirus B (CVB) compositions and methods of use of such compositions to induce an immune response to CVB in an individual.

### BRIEF DESCRIPTION OF THE DRAWINGS

FIG. 1 provides an amino acid sequence of a CVB4-encoded polyprotein (SEQ ID NO:1).

FIG. 2 depicts viral neutralizing titer (VNT) to CVB1 through CVB5 in serum of vaccinated human subjects at one month after the last dose.

FIG. 3 depicts peak VNT for each CVB serotype (CVB1-CVB5).

FIG. 4-13 depict VNT to CVB1 (FIG. 4 and FIG. 5), CVB2 (FIG. 6 and FIG. 7), CVB3 (FIG. 8 and FIG. 9), CVB4 (FIG. 10 and FIG. 11), and CBV5 (FIG. 12 and FIG. 13) in serum of vaccinated human subjects. The subjects were either seropositive for VNT to the indicated CVB serotype before vaccination (FIGS. 4, 6, 8, 10, and 12) or seronegative before vaccination (FIGS. 5, 7, 9, 11, and 13).

FIG. 14 depicts CVB-neutralizing antibody levels in baseline seronegative subjects following vaccination with a CVB composition.

FIG. 15 depicts enterovirus-specific absolute IgG levels in serum of subjects following vaccination with a CVB composition. Data include serum levels during the vaccination period and up to 6 months after the last dose. Data are represented as enzyme immunoassay units (EIU).

FIG. 16 depicts enterovirus-specific IgG levels in serum of subjects following vaccination with a CVB composition represented as fold increases in enterovirus-specific IgG levels over the levels measured at baseline for each individual.

FIG. 17 depicts the CVB-neutralizing antibody titers in all subjects (baseline seropositive and baseline seronegative) in serum of subjects following vaccination with a CVB composition. Data includes titers during the vaccination period and up to 6 months after the last dose. Titers are represented in a logarithmic (log 2) scale.

FIG. 18 depicts VNT in the serum of baseline seronegative individuals following vaccination with a CVB composition.

### DEFINITIONS

The terms “individual” and “patient” are used interchangeably herein to refer to an individual (e.g., a human) being treated using a method of the present disclosure.

“Treating,” and “treatment,” as used herein, refers to the treatment of the disease or condition of interest in a mammal, e.g., a human, having the disease or condition of interest, and includes, for example: (i) inhibiting or decreasing the severity of the disease or condition, or one or more symptoms thereof, e.g., arresting or slowing development or progression of the disease or condition, and/or ameliorating one or more symptoms; (ii) relieving the disease or condition, i.e., causing regression of the disease or condition, or one more symptoms thereof; and/or (iii) stabilizing the disease or condition.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “neutralizing antibody” or “viral neutralizing antibody” (VNT) is meant an antibody which either is purified from, or is present in, serum and which recognizes a specific antigen (e.g., a CVB-encoded polypeptide) and inhibits the infectivity of CVB and the effect(s) of the CVB in the host (e.g., a human).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a coxsackievirus B (CVB)” includes a plurality of such CVBs and reference to “the inactivated viral particle (IVP)” includes reference to one or more IVPs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides coxsackievirus B (CVB) compositions and methods of use of such compositions to induce an immune response to CVB in an individual.

### Compositions

The present disclosure provides CVB compositions, where such compositions are also referred to herein as “immunogenic compositions.” A CVB composition of the present disclosure can induce an immune response in an individual to one or more CVB serotypes. A CVB composition of the present disclosure can include whole CVB or portions of CVB. Thus, the term “CVB composition” includes: a) inactivated viral particles (IVP); b) a CVB viral-like particle (VLP); c) a CVB subunit (e.g., one or more CVB polypeptides); and d) one or more nucleic acids comprising nucleotide sequences encoding one or more CVB polypeptides. CVB-encoded polypeptides include VP1, VP2, VP3, VP4, 2A, 2B, 2C, 3A, 3B, 3C, and 3D. A “CVB composition” can comprise one or more CVB-encoded polypeptides, or a nucleic acid comprising a nucleotide sequence encoding one or more CVB-encoded polypeptides. Unless explicitly indicated otherwise, a composition comprising “CVB” refers to a composition comprising CVB IVPs, CVB VLPs, one or more CVB polypeptides, or nucleic acid(s) comprising nucleotide sequences encoding one or more CVB polypeptides.

Coxsackie B virus (CVB; also referred to herein and in the literature as “Coxsackie B virus” or “CBV”) is a group of 6 serotypes of Coxsackievirus (CVB1-CVB6). In some cases, a composition of the present disclosure includes CVB (in the form of CVB IVPs, CVB VLPs, CVB polypeptides, or nucleic acid(s) comprising nucleotide sequences encoding one or more CVB polypeptides) of all 6 serotypes; i.e., in some cases, a composition of the present disclosure in need thereof includes CVB1, CVB2, CVB3, CVB4, CVB5, and CVB6. In some cases, a composition of the present disclosure in need thereof includes only 5 serotypes. For example, in some cases, a composition of the present disclosure includes CVB1, CVB2, CVB3, CVB4, and CVB5, but not CVB6. In some cases, a composition of the present disclosure includes only a single CVB serotype. For example, in some cases, a composition of the present disclosure includes only CVB1. As another example, in some cases, a composition of the present disclosure includes only CVB2. As another example, in some cases, a composition of the present disclosure includes only CVB3. As another example, in some cases, a composition of the present disclosure includes only CVB4. As another example, in some cases, a composition of the present disclosure includes only CVB5. In some cases, a composition of the present disclosure includes CVB of only 2 different serotypes (e.g., CVB1 and CVB2; CVB1 and CVB3; CVB1 and CVB4; CVB1 and CVB5; CVB2 and CVB3; CVB2 and CVB4; CVB2 and CVB5; CVB3 and CVB4; CVB3 and CVB5; or CVB4 and CVB5) and does not include CVB of any other serotype. In some cases, a composition of the present disclosure includes CVB of only 3 different serotypes (e.g., CVB1, CVB2, and CVB3; CVB1, CVB2, and CVB4; CVB1, CVB2, and CVB5; CVB1, CVB3, and CVB4; CVB1, CVB3, and CVB5; CVB2, CVB3, and CVB4; CVB2, CVB3, and CVB5; CVB3, CVB4, and CVB5) and does not include CVB of any other serotype. In some cases, a composition of the present disclosure includes CVB of only 4 different serotypes (e.g., the composition may exclude: CVB3 and CVB6; CVB4 and CVB6; CVB5 and CVB6; other any other combination of 2 CVB serotypes).

In some cases, a composition of the present disclosure does not include any enterovirus other than CVB. For example, in some cases, a composition of the present disclosure does not include any whole virus, inactivated virus, viral protein (e.g., viral subunit), or nucleic acid encoding a viral protein, of an enterovirus other than CVB.

In some cases, a composition of the present disclosure comprises CVB of one or more serotypes, where the CVB is in “inactivated” form, which means that the infectivity of the virus has been reduced or eliminated. The term as used herein also includes viruses that are replication defective. A replication defective virus is a virus defective for a constitutive protein. Such a virus can enter the cell, deliver the partial genome, translate the proteins encoded and replicate the genome but cannot encapsidate new particles. Thus, it cannot spread to neighboring cells or tissue.

Inactivated CVB (CVB IVP) may be produced by propagating the virus in cell culture and by purifying it from infected cells and culture media by high-speed centrifugation in a density gradient formed by sucrose or other high-density media. Alternatively, the virus may be purified by chromatography. The infectivity of the purified viruses can be destroyed by inactivating the viruses by chemical treatment (e.g. formalin inactivation like that used to produce inactivated polio virus vaccine), irradiation or heat treatment. Replication defective viruses may be prepared by physical or genetic inactivation e.g., by deleting a structural gene in the viral genome, and producing a complementing cell line constitutively expressing the protein encoded by the gene deleted in order to replicate the defective virus. In some cases, a composition of the present disclosure comprises formalin-inactivated CVB IVPs.

In some cases, a composition of the present disclosure comprises CVB of from two to five serotypes, wherein the CVB is the form of IVPs, CVB VLPs, one or more CVB polypeptides, or one or more nucleic acids comprising a nucleotide sequence(s) encoding the one or more CVB polypeptides, wherein the composition comprises two or more of:
a) CVB1 in an amount of from 2.0×10⁷ IVP to 2.5×10⁸ IVP;
b) CVB2 in an amount of from 1.0×10⁷ IVP to 1.5×10⁸ IVP;
c) CVB3 in an amount of from 1.0×10⁸ IVP to 1.5×10⁹ IVP;
d) CVB4 in an amount of from 2.0×10⁷ IVP to 2.5×10⁸ IVP; and
e) CVB5 in an amount of from 4.0×10⁷ IVP to 4.0×10⁸ IVP,
or an equivalent amount of VLP, one or more CVB polypeptides, or one or more nucleic acids comprising nucleotide sequences encoding the one or more CVB polypeptides, where the amounts of CVB are present in a volume of from about 75 μL to about 600 μL (e.g., from about 75 μL to about 100 μL, from about 100 μL to about 150 μL, from about 150 μL to about 200 μL, from about 200 μL to about 250 μL, from about 250 μL to about 300 μL, from about 300 μL to about 350 μL, from about 350 μL to about 400 μL, from about 400 μL to about 450 μL, from about 450 μL to about 500 μL, from about 500 μL to about 550 μL, or from about 550 μL to about 600 μL). In some cases, the composition comprises the amounts of CVB in a volume of 100 μL. In some cases, the composition comprises the amounts of CVB in a volume of 500 μL. In some cases, the composition does not include CBV6.

In some cases, a composition of the present disclosure comprises CVB of from two to five serotypes, wherein the CVB is the form of IVPs, CVB VLPs, one or more CVB polypeptides, or one or more nucleic acids comprising a nucleotide sequence(s) encoding the one or more CVB polypeptides, wherein the composition comprises two or more of:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides. The amounts of CVB can be in a volume of from about 75 μL to about 150 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 L, from about 110 μL to about 120 μL, from about 120 μL to about 130 μL, from about 130 μL to about 140 μL, or from about 140 μL to about 150 μL. In some cases, the composition comprises the amounts of CVB in a volume of 100 μL. In some cases, the composition does not include CBV6.

In some cases, a composition of the present disclosure comprises CVB of from two to five serotypes, wherein the CVB is the form of IVPs, CVB VLPs, one or more CVB polypeptides, or one or more nucleic acids comprising a nucleotide sequence(s) encoding the one or more CVB polypeptides, wherein the composition comprises two or more of:
a) CVB1 in an amount of from 1.0×10⁸ IVP to 2.5×10⁸ IVP;
b) CVB2 in an amount of from 5.0×10⁷ IVP to 1.5×10⁸ IVP;
c) CVB3 in an amount of from 5.0×10⁸ IVP to 1.5×10⁹ IVP;
d) CVB4 in an amount of from 1.0×10⁸ IVP to 2.5×10⁸ IVP; and
e) CVB5 in an amount of from 2.0×10⁸ IVP to 4.0×10⁸ IVP,
or an equivalent amount of VLP, one or more CVB polypeptides, or one or more nucleic acids comprising nucleotide sequences encoding the one or more CVB polypeptides. The amounts of CVB can be in a volume of from about 400 μL to about 450 μL, from about 450 μL to about 500 μL, from about 500 μL to about 550 μL, or from about 550 μL to about 600 μL. In some cases, the composition comprises the amounts of CVB in a volume of 500 μL. In some cases, the composition does not include CBV6.

In some cases, a composition of the present disclosure comprises:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides. The amounts of CVB can be in a volume of from about 75 μL to about 150 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 L, from about 110 μL to about 120 μL, from about 120 μL to about 130 μL, from about 130 μL to about 140 μL, or from about 140 μL to about 150 μL. In some cases, the composition comprises the amounts of CVB in a volume of 100 μL. In some cases, the composition does not include CBV6.

In some cases, a composition of the present disclosure comprises:
a) CVB1 in an amount of from 3.0×10⁷ IVP to 4.0×10⁷ IVP;
b) CVB2 in an amount of from 1.0×10⁷ IVP to 2.0×10⁷ IVP;
c) CVB3 in an amount of from 1.0×10⁸ IVP to 2.0×10⁸ IVP;
d) CVB4 in an amount of from 3.0×10⁷ IVP to 4.0×10⁷ IVP; and
e) CVB5 in an amount of from 6.0×10⁷ IVP to 6.0×10⁷ IVP,
or an equivalent amount of VLP, one or more CVB polypeptides, or one or more nucleic acids comprising nucleotide sequences encoding the one or more CVB polypeptides. The amounts of CVB can be in a volume of from about 75 μL to about 150 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 L, from about 110 μL to about 120 μL, from about 120 μL to about 130 μL, from about 130 μL to about 140 μL, or from about 140 μL to about 150 μL. In some cases, the composition comprises the amounts of CVB in a volume of 100 μL. In some cases, the composition does not include CBV6.

In some cases, a composition of the present disclosure comprises:
a) CVB1: 3.3×10⁷ IVP;
b) CVB2: 1.1×10⁷ IVP;
c) CVB3 1.5×10⁸ IVP;
d) CVB4: 3.2×10⁷ IVP; and
e) CVB5: 6.3×10⁷ IVP,
or an equivalent amount of VLP, one or more CVB polypeptides, or one or more nucleic acids comprising nucleotide sequences encoding the one or more CVB polypeptides. The amounts of CVB can be in a volume of from about 75 μL to about 150 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 L, from about 110 μL to about 120 μL, from about 120 μL to about 130 μL, from about 130 μL to about 140 μL, or from about 140 μL to about 150 μL. In some cases, the composition comprises the amounts of CVB in a volume of 100 μL. In some cases, the composition does not include CBV6.

In some cases, a composition of the present disclosure comprises:
a) CVB1: 1.6×10⁸ IVP;
b) CVB2: 5.5×10⁷ IVP;
c) CVB3: 4.5×10⁹ IVP;
d) CVB4: 1.6×10⁸ IVP; and
e) CVB5: 4.8×10⁸ IVP, or an equivalent amount of VLP, one or more CVB polypeptides, or one or more nucleic acids comprising nucleotide sequences encoding the one or more CVB polypeptides. The amounts of CVB can be in a volume of from about 400 μL to about 450 μL, from about 450 μL to about 500 μL, from about 500 μL to about 550 μL, or from about 550 μL to about 600 μL. In some cases, the composition comprises the amounts of CVB in a volume of 500 μL. In some cases, the composition does not include CBV6.

In some cases, a composition of the present disclosure comprises:
a) CVB1: 1.6×10⁸ IVP;
b) CVB2: 5.5×10⁷ IVP;
c) CVB3: 7.5×10⁸ IVP;
d) CVB4: 1.6×10⁸ IVP; and
e) CVB5: 3.1×10⁸ IVP, or an equivalent amount of VLP, one or more CVB polypeptides, or one or more nucleic acids comprising nucleotide sequences encoding the one or more CVB polypeptides. The amounts of CVB can be in a volume of from about 400 μL to about 450 μL, from about 450 μL to about 500 μL, from about 500 μL to about 550 μL, or from about 550 μL to about 600 μL. In some cases, the composition comprises the amounts of CVB in a volume of 500 μL. In some cases, the composition does not include CBV6.

In some cases, a composition of the present disclosure comprises:
a) CVB1 in an amount of from 2.0×10⁷ IVP to 2.5×10⁸ IVP;
b) CVB2 in an amount of from 1.0×10⁷ IVP to 1.5×10⁸ IVP;
c) CVB3 in an amount of from 1.0×10⁸ IVP to 1.5×10⁹ IVP;
d) CVB4 in an amount of from 2.0×10⁷ IVP to 2.5×10⁸ IVP; and
e) CVB5 in an amount of from 4.0×10⁷ IVP to 4.0×10⁸ IVP,
or an equivalent amount of VLP, one or more CVB polypeptides, or one or more nucleic acids comprising nucleotide sequences encoding the one or more CVB polypeptides. The amounts of CVB can be in a volume of from about 75 μL to about 600 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 L, from about 110 μL to about 120 μL, from about 120 μL to about 130 μL, from about 130 μL to about 140 μL, from about 140 μL to about 150 μL, from about 150 μL to about 200 μL, from about 200 μL to about 250 μL, from about 250 μL to about 300 μL, from about 300 μL to about 350 μL, from about 350 μL to about 400 μL, from about 400 μL to about 450 μL, from about 450 μL to about 500 μL, from about 500 μL to about 550 μL, or from about 550 μL to about 600 μL). In some cases, the composition does not include CBV6.

In some cases, a composition of the present disclosure comprises two or more of:
a) CVB1 in an amount of from about 1 μg protein to about 50 μg protein (e.g., from about 1 μg to 2 μg, from 2 μg to 3 μg, from 3 μg to 5 μg, from 5 μg to 10 μg, from 10 μg to 15 μg, from g to 20 μg, from 20 μg to 25 μg, from 25 μg to 30 μg, from 30 μg to 35 μg, from 35 μg to 40 μg, from 40 μg to 45 μg, or from 45 μg to 50 μg);
b) CVB2 in an amount of from about 1 μg protein to about 50 μg protein (e.g., from about 1 μg to 2 μg, from 2 μg to 3 μg, from 3 μg to 5 μg, from 5 μg to 10 μg, from 10 μg to 15 μg, from g to 20 μg, from 20 μg to 25 μg, from 25 μg to 30 μg, from 30 μg to 35 μg, from 35 μg to 40 μg, from 40 μg to 45 μg, or from 45 μg to 50 μg);
c) CVB3 in an amount of from about 1 μg protein to about 50 μg protein (e.g., from about 1 μg to 2 μg, from 2 μg to 3 μg, from 3 μg to 5 μg, from 5 μg to 10 μg, from 10 μg to 15 μg, from g to 20 μg, from 20 μg to 25 μg, from 25 μg to 30 μg, from 30 μg to 35 μg, from 35 μg to 40 μg, from 40 μg to 45 μg, or from 45 μg to 50 μg);
d) CVB4 in an amount of from about 1 μg protein to about 50 μg protein (e.g., from about 1 μg to 2 μg, from 2 μg to 3 μg, from 3 μg to 5 μg, from 5 μg to 10 μg, from 10 μg to 15 μg, from g to 20 μg, from 20 μg to 25 μg, from 25 μg to 30 μg, from 30 μg to 35 μg, from 35 μg to 40 μg, from 40 μg to 45 μg, or from 45 μg to 50 μg); and
e) CVB5 in an amount of from about 1 μg protein to about 50 μg protein (e.g., from about 1 μg to 2 μg, from 2 μg to 3 μg, from 3 μg to 5 μg, from 5 μg to 10 μg, from 10 μg to 15 μg, from g to 20 μg, from 20 μg to 25 μg, from 25 μg to 30 μg, from 30 μg to 35 μg, from 35 μg to 40 μg, from 40 μg to 45 μg, or from 45 μg to 50 μg), for a total amount of protein of from about 5 μg to about 250 μg. The amounts of protein can be in a volume of from about 75 μL to about 600 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 μL, from about 110 μL to about 120 μL, from about 120 μL to about 130 μL, from about 130 μL to about 140 μL, from about 140 μL to about 150 μL, from about 150 μL to about 200 μL, from about 200 μL to about 250 μL, from about 250 μL to about 300 μL, from about 300 μL to about 350 μL, from about 350 μL to about 400 μL, from about 400 μL to about 450 μL, from about 450 μL to about 500 μL, from about 500 μL to about 550 μL, or from about 550 μL to about 600 μL). In some cases, the composition does not include CBV6.

In some cases, a composition of the present disclosure comprises:
a) CVB1 in an amount of from about 2 μg protein to about 4 μg protein;
b) CVB2 in an amount of from about 1 μg protein to about 2.5 μg protein;
c) CVB3 in an amount of from about 2 μg protein to about 4 μg protein;
d) CVB4 in an amount of from about 1 μg protein to about 2 μg protein; and
e) CVB5 in an amount of from about 1 μg protein to about 2.5 μg protein, for a total amount of protein of about from about 8 μg protein to about 15 μg protein. The amounts of protein can be in a volume of from about 75 μL to about 150 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 μL, from about 110 μL to about 120 μL, from about 120 μL to about 130 μL, from about 130 μL to about 140 μL, or from about 140 μL to about 150 μL). In some cases, the composition comprises the amounts of CVB in a volume of 100 μL. In some cases, the composition does not include CBV6.

In some cases, a composition of the present disclosure comprises:
a) CVB1 in an amount of 2.8 μg protein;
b) CVB2 in an amount of 1.7 μg protein;
c) CVB3 in an amount of 2.7 μg protein;
d) CVB4 in an amount of 1.2 μg protein; and
e) CVB5 in an amount of 2.3 μg protein, for a total amount of protein of about 10.7 μg. The amounts of protein can be in a volume of from about 75 μL to about 150 μL (e.g., from about 75 μL to about 80 μL, from about 80 μL to about 85 μL, from about 85 μL to about 90 μL, from about 90 μL to about 110 μL, from about 95 μL to about 105 μL, from about 95 μL to about 100 μL, from about 100 μL to about 110 μL, from about 110 μL to about 120 μL, from about 120 μL to about 130 μL, from about 130 μL to about 140 μL, or from about 140 μL to about 150 μL. In some cases, the composition comprises the amounts of CVB in a volume of 100 μL. In some cases, the composition does not include CBV6.

In some cases, a composition of the present disclosure comprises:
a) CVB1 in an amount of from about 10 μg protein to about 20 μg protein;
b) CVB2 in an amount of from about 5 μg protein to about 12.5 μg protein;
c) CVB3 in an amount of from about 10 μg protein to about 20 μg protein;
d) CVB4 in an amount of from about 5 μg protein to about 10 μg protein; and
e) CVB5 in an amount of from about 5 μg protein to about 12.5 μg protein, for a total amount of protein of about from about 35 μg protein to about 75 μg protein. The amounts of CVB can be in a volume of from about 400 μL to about 450 μL, from about 450 μL to about 500 μL, from about 500 μL to about 550 μL, or from about 550 μL to about 600 μL. In some cases, the composition comprises the amounts of CVB in a volume of 500 μL. In some cases, the composition does not include CBV6.

In some cases, a composition of the present disclosure comprises:
a) CVB1 in an amount of 14 μg protein;
b) CVB2 in an amount of 8.5 μg protein;
c) CVB3 in an amount of 13.5 μg protein;
d) CVB4 in an amount of 6 μg protein; and
e) CVB5 in an amount of 11.5 μg protein, for a total amount of protein of 53.5 μg. The amounts of CVB can be in a volume of from about 400 μL to about 450 μL, from about 450 μL to about 500 μL, from about 500 μL to about 550 μL, or from about 550 μL to about 600 μL. In some cases, the composition comprises the amounts of CVB in a volume of 500 μL. In some cases, the composition does not include CBV6.

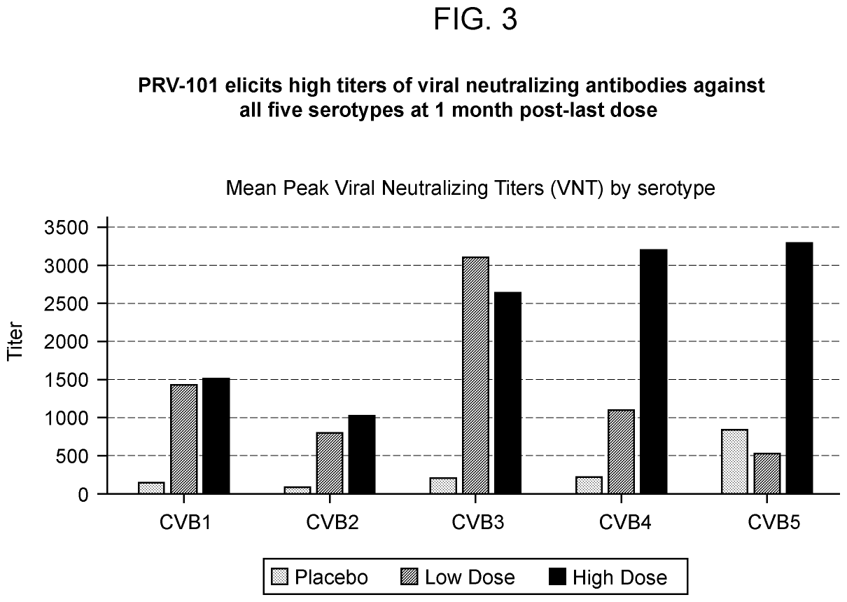

A composition of the present disclosure, when administered to an individual, induces a viral neutralizing antibody titer (VNT) of from 1/8 to 1/64,000, as determined by a VNT assay. In some cases, such VNT is a peak VNT (e.g., as depicted in FIG. 3). In some cases, a composition of the present disclosure, when administered to an individual, induces a VNT of from about 1/2,000 to about 1/4,000, as determined by a VNT assay. In some cases, a composition of the present disclosure, when administered to an individual, induces a VNT of from about 1/10 to about 1/500, as determined by a VNT assay. In some cases, a composition of the present disclosure, when administered to an individual, induces a VNT of from about 1/500 to about 1/1,000, as determined by a VNT assay. In some cases, a composition of the present disclosure, when administered to an individual, induces a VNT of from about 1/1,000 to about 1/2,000, as determined by a VNT assay. In some cases, a composition of the present disclosure, when administered to an individual, induces a VNT of from about 1/2,000 to about 1/4,000, as determined by a VNT assay. In some cases, a composition of the present disclosure, when administered to an individual, induces a VNT of from about 1/4,000 to about 1/8,000, as determined by a VNT assay. In some cases, a composition of the present disclosure, when administered to an individual, induces a VNT of from about 1/8,000 to about 1/10,000, as determined by a VNT assay. In some cases, a composition of the present disclosure, when administered to an individual, induces a VNT of from about 1/10,000 to about 1/20,000, as determined by a VNT assay.

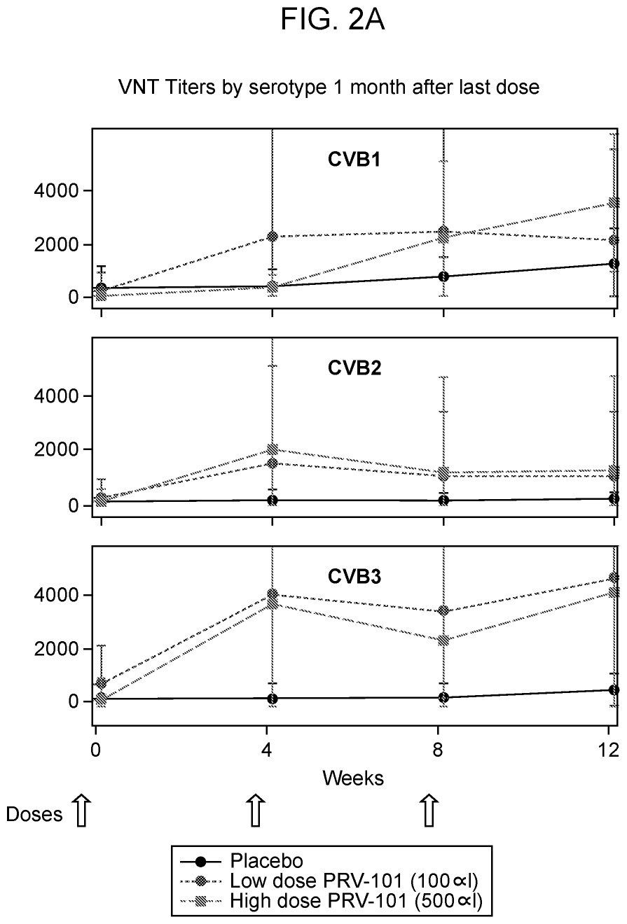

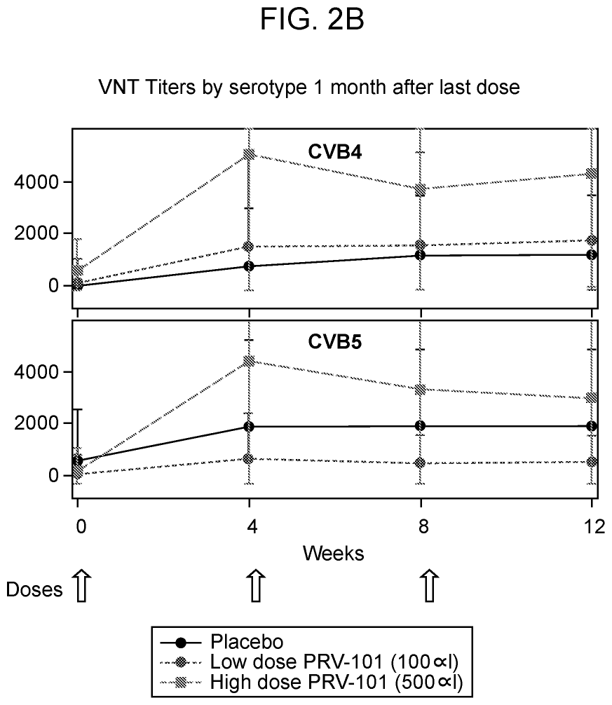

As used herein, “viral neutralizing antibody titer” refers to the highest dilution of the sample which still neutralizes the infectivity of the virus in cell culture. The viral neutralizing titer (VNT) is calculated as the last serum dilution that reduces the number of plaques by at least 80% using a plaque reduction assay. See, e.g., Boone and Albrecht (1983) J. Virol. Methods 6:193. Thus, e.g., a VNT of 1/2,000 refers to the amount of antibody in a serum sample wherein, when the sample is diluted by more than 1/2,000 (“a 2,000 VNT”), no longer neutralizes CVB in the sample. For example, in FIG. 2 and FIG. 3, “2,000” means a 2000× dilution (referred to as “1/2,000” or a “2,000 VNT”) is the highest dilution that reduces infection of a cell in culture by at least 80%; and “4,000” means a 4000× dilution (referred to as “1/4,000” or a “4,000 VNT”) is the highest dilution that reduces infection of a cell in culture by at least 80%.

In some cases, a single dose of a subject composition is administered to an individual. In some cases, the VNT against CVB in an individual at 14 days post-administration of the single dose of the composition is increased by 8- to 10-fold relative to a control. In some cases, the VNT against CVB in an individual at 14 days post-administration of the single dose of the composition is increased by 3- to 10-fold relative to a control. A control refers to: i) the VNT against CVB in the individual before administration of the single dose of a composition of the present disclosure; or ii) a reference control level of VNT against CVB.

In some cases, a first dose of a subject composition is administered to an individual; and a second dose of a subject composition is administered to the individual at a time that is after the first dose (e.g., the second dose is administered from 7 days to 3 weeks, from 7 days to 2 weeks, from 2 weeks to 1 month, from 1 month to 2 months, from 2 months to 3 months, or from 3 months to 6 months, after the first dose). In some cases, a first dose of a subject composition is administered to an individual; and a second dose of a subject composition is administered to the individual 1 month after the first dose. In some cases, a first dose of a subject composition is administered to an individual; and a second dose of a subject composition is administered to the individual 2 months after the first dose. In some cases, a first dose of a subject composition is administered to an individual; and a second dose of a subject composition is administered to the individual 3 months after the first dose. In some cases, the VNT against CVB in an individual at 14 days post-administration of the second dose of the composition is increased by 8- to 10-fold relative to a control. In some cases, the VNT against CVB in an individual at 14 days post-administration of the second dose of the composition is increased by 3- to 10-fold relative to a control. A control refers to: i) the VNT against CVB in the individual before administration of the first dose of a composition of the present disclosure; or ii) a reference control level of VNT against CVB.

A neutralizing titer is produced by neutralizing antibody against CVB as measured in serum of the subject. In some cases, an effective dose of a composition of the present disclosure is sufficient to produce a viral neutralization titer (VNT) of at least 100. In some cases, an effective dose of a composition of the present disclosure is sufficient to produce a VNT of from 100 to 500. In some cases, an effective dose of a composition of the present disclosure is sufficient to produce a VNT of more than 500. For example, in some cases, an effective dose of a composition of the present disclosure is sufficient to produce a 500-1,000 VNT. As another example, in some cases, an effective dose of a composition of the present disclosure is sufficient to produce a 1000-10,000 VNT. In some cases, an effective dose of a composition of the present disclosure is sufficient to produce a 1000-2000, 1000-3000, 1000-4000, 1000-5000, 1000-6000, 1000-7000, 1000-8000, 1000-9000, 1000-10,000, 2000-3000, 2000-4000, 2000-5000, 2000-6000, 2000-7000, 2000-8000, 2000-9000, 2000-10,000, 3000-4000, 3000-5000, 3000-6000, 3000-7000, 3000-8000, 3000-9000, 3000-10,000, 4000-5000, 4000-6000, 4000-7000, 4000-8000, 4000-9000, 4000-10,000, 5000-6000, 5000-7000, 5000-8000, 5000-9000; 5000-10,000, 6000-7000, 6000-8000, 6000-9000, 6000-10,000, 7000-8000, 7000-9000, 7000-10,000, 8000-9000, 8000-10,000, or a 9000-10,000 VNT. In some cases, an effective dose of a composition of the present disclosure is sufficient to produce a 500, 1000, 1500, 2000, 2500, 3000, 3500, 4000, 4500, 5000, 5500, 6000, 6500, 7000, 7500, 8000, 8500, 9000, 9500, 10,000, 11000, 12,000, 13,000, 14,000, 15,000, 16,000, 17,000, 18,000, 19000, 20,000 or higher VNT. In some cases, VNT is produced 1-72 hours post administration. For example, VNTs may be produced 1-10, 1-20, 1-30, 1-40, 1-50, 1-60, 1-70, 1-72, 10-20, 10-30, 10-40, 10-50, 10-60, 10-70, 10-72, 20-30, 20-40, 20-50, 20-60, 20-70, 20-72, 30-40, 30-50, 30-60, 30-70, 30-72, 40-50, 40-60, 40-70, 40-72, 50-60, 50-70, 50-72, 60-70, 60-72, or 70-72 hours post administration. In some cases, VNTs may be produced 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 56, 47, 48, 49, 50, 51, 52, 53, 54, 55, 56, 57, 58, 59, 60, 61, 62, 63, 64, 65, 66, 67, 68, 69, 70, 71, or 72 hours post administration. In some cases, VNT is produced within 14 days of administration of a composition of the present disclosure. In some cases, VNT is produced within 5, 6, 7, 8, 9, 10, 11, 12, 13, or 14 days of administration of a composition of the present disclosure.

In some cases, a VNT of from 1/8 to 1/64,000 is maintained for a period of time of at least 24 weeks, at least 32 weeks, at least 40 weeks, at least 52 weeks, or more than 52 weeks, following administration of one or more doses of a CVB composition of the present disclosure. In some cases, the VNT comprises IgG isotype antibodies (e.g., the VNT comprises at least 50%, at least 60%, at least 75%, at least 85%, or more than 85%, IgG isotype antibodies). In some cases, the VNT comprises IgG isotype antibodies, IgM isotype antibodies, and IgA isotype antibodies.

VNT can be determined using a VNT assay (also referred to as a “plaque reduction assay”). The presence of neutralizing antibodies blocks the infectivity of the virus into cells. To carry out a VNT assay, serum obtained from an individual is diluted to various degrees. The diluted serum is mixed with a certain amount of infectious virus (e.g., 30-100 plaque forming units (PFU) of the virus, where PFU represents one infective virus particle) optimized for each virus serotype, and the mixture is incubated for a certain period of time (e.g., first for 60 min at 37° C. followed by overnight (approximately 18 hours) incubation) at room temperature. After the incubation period, the mixture is added to a monolayer of cells (e.g., green monkey kidney cells (GMK-AH-1 cells; RRID:CVCL_L878)) and the cell monolayer is incubated with the mixture for a certain period of time (e.g., for 40-48 hours). After this incubation, the number of virus-induced plaques on the monolayer is counted, e.g., after staining the cells or by microscopic examination of the cells. A mixture of virus and hyperimmune serum raised against the enterovirus serotype in question (e.g., serum raised in monkeys or horses) serves as a positive control for the specific neutralization of each serotype. Virus without serum serves as a negative control. The neutralizing titer (VNT) is calculated as the last serum dilution which reduces the number of plaques by at least 80%. See, e.g., Boone and Albrecht (1983) J. Virol. Methods 6:193.

A composition of the present disclosure may include whole CVB viruses, the infectivity of which has been inactivated, or subunit vaccines containing certain antigenic structures, proteins or peptides of the virus, or their combination (such as virus like particles), or fragments of viral RNA or cDNA encoding the whole virus or individual viral proteins or inactivated forms of the virus.

In some cases, a composition of the present disclosure comprises a component of a CVB. The “component” may be an immunogenic polypeptide of the virus such as a subunit thereof including a chimeric subunit, or a nucleic acid fragment such as part of the genome of the virus. The component may also be recombinantly or synthetically produced or modified.

Subunit vaccines may consist of purified viral proteins or recombinant viral proteins, synthetic peptides corresponding to viral antigenic epitopes, VLPs, or empty viral capsids, which are produced during infection but lack the viral genome. These subunit vaccines can be administered either as such or conjugated to haptens or carriers (e.g., ISCOM particles, chitosan, TLR agonists, biodegradable microparticles).

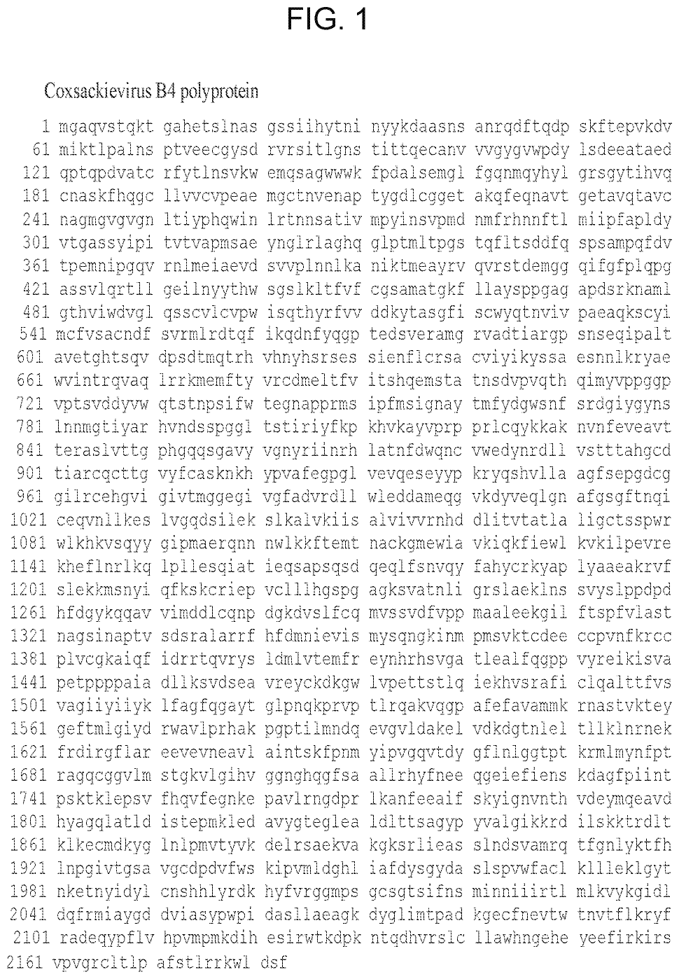

As noted above, a composition of the present disclosure can include a CVB subunit, e.g., one or more CVB polypeptides. CVB-encoded polypeptides include VP1, VP2, VP3, VP4, 2A, 2B, 2C, 3A, 3B, 3C, and 3D. For example, the composition can comprise one or more CVB polypeptides, where the one or more CVB polypeptides comprise an amino acid sequence having at least 75%, at least 80%, at least 85%, at least 90%, at least 95%, at least 98%, at least 99%, or 100%, amino acid sequence identity to the amino acid sequence for the whole virus polyprotein (for example the CVB4 polyprotein shown in FIG. 1) or any polypeptides derived from the precursor polyprotein. For example, the initial 73 amino acids of the CVB4 polyprotein in FIG. 1, forms the CBV4 VP4 component of the virus upon maturation of the polyprotein. The composition can comprise any CBV4 VP4 polypeptide comprising an amino acid sequence having at least 75%, at least 80%, at least 85%, at least 90%, at least 95%, at least 98%, at least 99%, or 100%, amino acid sequence identity with either the full amino acid sequence or with a polypeptide fragment of the mature form of CBV4 VP4. The VLP can be formed using full-length viral proteins, truncated forms of the full-length viral proteins, or fusion proteins that contain a part of the viral protein.

As noted above, a composition suitable for administration to an individual in need thereof can include one or more nucleic acids comprising a nucleotide sequence(s) encoding one or more CVB polypeptides. Suitable nucleic acids include recombinant expression vectors. In some cases, the one or more nucleic acids are recombinant expression vectors comprising one or nucleotide sequences encoding one or more CVB polypeptides. In some cases, the recombinant expression vector is a DNA or RNA molecule comprising a nucleotide sequence encoding a gene product (e.g., an RNA or a polypeptide) of interest. Recombinant expression vectors include viral expression vectors (e.g. viral vectors based on vaccinia virus; poliovirus; adenovirus, adeno-associated virus (AAV), a lentivirus, SV40, herpes simplex virus, a human immunodeficiency virus (HIV), a retrovirus, and the like). In some cases, the recombinant expression vector is a recombinant lentivirus vector. In some cases, the recombinant expression vector is a recombinant HIV vector. In some cases, the recombinant expression vector is a recombinant adenovirus vector. In some cases, the recombinant expression vector is a recombinant AAV vector. In some cases, the nucleotide sequence(s) encoding the one or more CVB polypeptides is operably linked to one or more transcriptional control elements, such as a promoter. In some cases, the nucleotide sequence encoding the gene product of interest is operably linked to a promoter that is functional in a mammalian cell, e.g., a muscle cell, an epithelial cell, a dendritic cell, an antigen-presenting cell, and the like.

Suitable nucleic acids include mRNA. Thus, in some cases, a composition suitable for administering to an individual in need thereof comprises one or more RNA molecules (e.g., mRNA) comprising one or more nucleotide sequences encoding one or more CVB polypeptides. In some cases, the one or more RNA molecules comprise at least one 5′ cap structure and/or a 5′ untranslated region (5′ UTR). In some cases, the one or more RNA molecules also comprise a 3′ UTR and/or a 3′ tailing sequence (e.g., a poly(adenosine) (poly-A) sequence). Examples of 5′ cap structures include inosine, N1-methyl-guanosine, 2′fluoro-guanosine, 7-deaza-guanosine, 8-oxo-guanosine, 2-amino-guanosine, LNA-guanosine, and 2-azido-guanosine. In some cases, the one or more RNA molecules comprises one or more of: a nucleoside base modification, a sugar modification, and a backbone modification. In some cases, the composition comprises, in addition to the one or more RNA molecules, one or both of: a) a polymer (e.g., polyethylene glycol, polyglycolide, polyvinyl alcohol, polyvinyl pyrrolidone, polylactide, poly(lactide-co-glycolide) (PLGA), polycaprolactone, polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, polyethyleneimine, polyamidoamine (PAMAM) dendrimer, and block copolymer poly(ethylene glycol)-block-poly(lactic-co-glycolic acid) (PEG-b-PLGA)); and b) a lipid.

In some cases, a composition suitable for administering to an individual in need thereof comprises one or more RNA molecules (e.g., mRNA) comprising one or more nucleotide sequences encoding one or more CVB polypeptides. In some cases, the one or more RNA molecules are administered to an individual in need thereof in an amount such that the level of the encoded CVB polypeptide(s) in the serum of the individual is at least 50 pg/mL at least 2 hours after administration. In some cases, the one or more RNA molecules are administered to an individual in need thereof in an amount such that the level of the encoded CVB polypeptide(s) in the serum of the individual remains above 50 μg/mL for at least 72 hours after administration.

In some cases, each of the one or more mRNAs is administered in an amount of from about 1 μg to about 200 μg, e.g., from about 1 μg to about 5 μg, from about 5 μg to about 10 μg, from about 10 μg to about 15 μg, from about 15 μg to about 20 μg, from about 20 μg to about 25 μg, from about 25 μg to about 30 μg, from about 30 μg to about 40 μg, from about 40 μg to about 50 μg, from about 50 μg to about 60 μg, from about 60 μg to about 70 μg, from about 70 μg to about 80 μg, from about 80 μg to about 90 μg, from about 90 μg to about 100 μg, from about 100 μg to about 125 μg, from about 125 μg to about 150 μg, from about 150 μg to about 175 μg, or from about 175 μg to about 200 μg.

For example, in some cases, a composition of the present disclosure includes nucleic acids (e.g., mRNAs) encoding CVB1, CVB2, CVB3, CVB4, and CVB5, but not CVB6, where each of the mRNAs is present in the composition in an amount of from about 1 μg to about 200 μg, e.g., from about 1 μg to about 5 μg, from about 5 μg to about 10 μg, from about 10 μg to about 15 μg, from about g to about 20 μg, from about 20 μg to about 25 μg, from about 25 μg to about 30 μg, from about 30 g to about 40 μg, from about 40 μg to about 50 μg, from about 50 μg to about 60 μg, from about 60 μg to about 70 μg, from about 70 μg to about 80 μg, from about 80 μg to about 90 μg, from about 90 μg to about 100 μg, from about 100 μg to about 125 μg, from about 125 μg to about 150 μg, from about 150 μg to about 175 μg, or from about 175 μg to about 200 μg.

In some cases, a composition of the present disclosure includes a nucleic acid (e.g., an mRNA) encoding polypeptides of only a single CVB serotype. For example, in some cases, a composition of the present disclosure includes nucleic acids encoding only CVB1 polypeptides. As another example, in some cases, a composition of the present disclosure includes nucleic acids encoding only CVB2 polypeptides. As another example, in some cases, a composition of the present disclosure includes nucleic acids encoding only CVB3 polypeptides. As another example, in some cases, a composition of the present disclosure includes nucleic acids encoding only CVB4 polypeptides. As another example, in some cases, a composition of the present disclosure includes nucleic acids encoding only CVB5 polypeptides. In some cases, the nucleic acid encoding only polypeptides of a single CVB serotype is mRNA, and the mRNA is present in the composition in an amount of from about 1 μg to about 200 μg, e.g., from about 1 μg to about 5 μg, from about 5 μg to about 10 μg, from about 10 μg to about 15 μg, from about 15 μg to about 20 μg, from about 20 μg to about 25 μg, from about 25 μg to about 30 μg, from about 30 μg to about 40 μg, from about 40 μg to about 50 μg, from about 50 μg to about 60 μg, from about 60 μg to about 70 μg, from about 70 μg to about 80 μg, from about 80 μg to about 90 μg, from about 90 μg to about 100 μg, from about 100 μg to about 125 μg, from about 125 μg to about 150 μg, from about 150 μg to about 175 μg, or from about 175 μg to about 200 μg.

In some cases, a composition of the present disclosure includes nucleic acids (e.g., mRNA) encoding CVB polypeptides of only 2 different serotypes (e.g., CVB1 and CVB2; CVB1 and CVB3; CVB1 and CVB4; CVB1 and CVB5; CVB2 and CVB3; CVB2 and CVB4; CVB2 and CVB5; CVB3 and CVB4; CVB3 and CVB5; or CVB4 and CVB5) and does not include a nucleic acid encoding CVB polypeptides of any other serotype. In some cases, the nucleic acids encoding polypeptides of only two CVB serotypes are mRNAs, and each of the mRNAs are present in the composition in an amount of from about 1 μg to about 200 μg, e.g., from about 1 μg to about 5 μg, from about 5 μg to about 10 μg, from about 10 μg to about 15 μg, from about 15 μg to about 20 μg, from about 20 μg to about 25 μg, from about 25 μg to about 30 μg, from about 30 μg to about 40 μg, from about 40 μg to about 50 μg, from about 50 μg to about 60 μg, from about 60 μg to about 70 μg, from about 70 μg to about 80 μg, from about 80 μg to about 90 μg, from about 90 μg to about 100 μg, from about 100 μg to about 125 μg, from about 125 μg to about 150 μg, from about 150 μg to about 175 μg, or from about 175 μg to about 200 μg.

In some cases, a composition of the present disclosure includes nucleic acids (e.g., mRNA) encoding CVB polypeptides of only 3 different serotypes (e.g., CVB1, CVB2, and CVB3; CVB1, CVB2, and CVB4; CVB1, CVB2, and CVB5; CVB1, CVB3, and CVB4; CVB1, CVB3, and CVB5; CVB2, CVB3, and CVB4; CVB2, CVB3, and CVB5; CVB3, CVB4, and CVB5) and does not include nucleic acid encoding CVB polypeptides of any other serotype. In some cases, the nucleic acids encoding polypeptides of only 3 CVB serotypes are mRNAs, and each of the mRNAs are present in the composition in an amount of from about 1 μg to about 200 μg, e.g., from about 1 μg to about 5 μg, from about 5 μg to about 10 μg, from about 10 μg to about 15 μg, from about 15 μg to about 20 μg, from about g to about 25 μg, from about 25 μg to about 30 μg, from about 30 μg to about 40 μg, from about 40 g to about 50 μg, from about 50 μg to about 60 μg, from about 60 μg to about 70 μg, from about 70 μg to about 80 μg, from about 80 μg to about 90 μg, from about 90 μg to about 100 μg, from about 100 μg to about 125 μg, from about 125 μg to about 150 μg, from about 150 μg to about 175 μg, or from about 175 μg to about 200 μg.

In some cases, a composition of the present disclosure includes nucleic acids encoding CVB polypeptides of only 4 different serotypes (e.g., the composition may exclude nucleic acids encoding polypeptides of: CVB3 and CVB6; CVB4 and CVB6; CVB5 and CVB6; other any other combination of 2 CVB serotypes).

The above-described CVBs, subunits, VLPs, or nucleic acids can be formulated into a pharmaceutical composition, which in addition to the active ingredients that elicit immune stimulation may comprise pharmaceutically acceptable excipients, carriers, haptens and/or adjuvants. Excipients, carriers, haptens and adjuvants may include for example phenoxyethanol, magnesium chloride, sucrose, thiomersal, formaldehyde, phenol, antibiotics (preservatives) or aluminum salts, polymer microparticles, ISCOM particles, carrier proteins (e.g. cholera toxin), liposomes, protein micelles (haptens/adjuvants), or TLR agonists.

A CVB composition can include an adjuvant (e.g., an immunostimulating amount of an adjuvant). A CVB composition can include an immune-stimulating amount of an adjuvant. Examples of known suitable adjuvants that can be used in humans include, but are not necessarily limited to, alum, aluminum phosphate, aluminum hydroxide, MF59 (4.3% w/v squalene, 0.5% w/v Tween 80™, 0.5% w/v Span 85), CpG-containing nucleic acid (where the cytosine is unmethylated), QS21, monophosphoryl lipid A (MPL), 3-Q-desacyl-4′-monophosphoryl lipid A (3DMPL), extracts from Aquilla, immune-stimulating complexes (ISCOMS; complexes of cholesterol, phospholipids, and Quillaja saponins), LT/CT mutants, poly(D,L-lactide-co-glycolide) (PLG) microparticles, Quil A, interleukins, and the like. For experimental animals, one can use Freund's incomplete adjuvant, or Freund's complete adjuvant. Also suitable for use are N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria: monophosphoryl lipid A, trehalose dimycolate and cell wall skeleton (MPL+TDM+CWS) in a 2% squalene/Tween 80 emulsion. As another example, an adjuvant can include another virus (other than CVB), either in combination with a composition of the present disclosure or as a carrier of CVB proteins displayed by the non-CVB virus.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (see, e.g., WO 90/14837), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (Detox™); (2) saponin adjuvants, such as QS21 or Stimulon™ (Cambridge Bioscience, Worcester, Mass.; a purified extract of Quillaja saponaria) may be used or particles generated therefrom such as ISCOMs (immunostimulating complexes), which ISCOMS may be devoid of additional detergent e.g. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO 00/56358; (6) combinations of 3dMPL with, for example, QS21 and/or oil-in-water emulsions (see, e.g. EP-A-0835318, EP-A-0735898, EP-A-0761231); (7) oligonucleotides comprising a CpG motif containing at least one CG dinucleotide, where the cytosine is unmethylated (see, e.g., WO 96/02555, WO 98/16247, WO 98/18810, WO 98/40100, WO 98/55495, WO 98/37919 and WO 98/52581); (8) a polyoxyethylene ether or a polyoxyethylene ester (see, e.g. WO 99/52549); (9) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (10) a saponin and an immunostimulatory oligonucleotide (e.g. a CpG oligonucleotide) (WO 00/62800); (11) an immunostimulant and a particle of metal salt (see, e.g. WO 00/23105); (12) a saponin and an oil-in-water emulsion (see e.g. WO 99/11241); (13) a saponin (e.g. QS21)+3dMPL+IM2 (optionally including a sterol) (see, e.g. WO 98/57659); (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc. Also suitable for use is Matrix-M™; Matrix-M™ is an adjuvant that comprises 40 nm nanoparticles comprising Quillaja saponins, cholesterol, and phospholipid. Adjuvants suitable for administration to a human are of particular interest. In some cases, the adjuvant is one that enhances a CD4⁺ T helper response to the immunogen. Also suitable for use is a poly inosine:cytosine (poly I:C) nucleic acid. Poly I:C is a synthetic double-stranded RNA Also suitable for use is a cyclic dinucleotide activator of the STING pathway. Examples of suitable cyclic dinucleotide adjuvants include, but are not limited to: 1) bis-(3′,5′)-cyclic dimeric adenosine monophosphate (c-di-AMP); 2) bis-(3′,5′)-cyclic dimeric guanosine monophosphate (c-di-GMP); and bis-(3′,5′)-cyclic dimeric inosine monosphosphate (c-di-IMP). Also suitable for use is poly(I:C).

The effectiveness of an adjuvant may be determined by one or more of: i) measuring the amount of antibodies directed against the immunogenic antigen or antigenic epitope thereof (e.g., using a VNT assay as described above); ii) measuring a cytotoxic T lymphocyte response to the antigen; and iii) measuring a helper T cell response to the antigen.

A composition of the present disclosure may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

In some cases, composition of the present disclosure is a liquid composition. Thus, the present disclosure provides compositions (e.g., liquid compositions, including pharmaceutical compositions) comprising CVB, as described above. In some cases, a composition of the present disclosure comprises: a) CVB, as described above; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) CVB, as described above; and b) saline (e.g., 0.9% NaCl), where the composition is sterile and is free of detectable pyrogens and/or other toxins.

### Methods of Inducing an Immune Response

The present disclosure provides methods of inducing an immune response to CVB in an individual. The methods comprise administering to an individual in need thereof an effective amount of a composition of the present disclosure. A composition of the present disclosure is referred to below as an “immunogenic composition.” The present disclosure provides methods of reducing the likelihood that an individual will develop an acute CVB infection. The methods comprise administering to an individual in need thereof an effective amount of a composition of the present disclosure. The present disclosure provides methods of reducing the likelihood that an individual will develop a CVB infection-associated disease and/or symptoms of a CVB infection. The methods comprise administering to an individual in need thereof an effective amount of a composition of the present disclosure.

An immunogenic composition as described above can be administered parenterally by injection (e.g., intramuscularly or subcutaneously), orally, perorally, intradermally, transcutaneously, sublingually, intranasally, nasopharyngeally, via inhalation, or per rectum.

In some cases, a prime-boost vaccine protocol is used. In some cases, a first (priming) immunogenic composition is administered, and, after a time, a second (booster) immunogenic composition is administered. A second immunogenic composition can be administered at a time period of from 1 day to 1 year following administration of the first immunogenic composition. For example, a second immunogenic composition can be administered at a time period of from 1 day to 1 week, from 1 week to 2 weeks, from 2 weeks to 1 month, from 1 month to 2 months, from 2 months to 6 months, or from 6 months to 1 year following administration of the first immunogenic composition.

A method of the present disclosure can induce an immune response in an individual to CVB. For example, a method of the present disclosure can induce an immune response in an individual to one or more of CVB1, CVB2, CVB3, CVB4, and CVB5. In some cases, a method of the present disclosure can induce an immune response in an individual to CVB1, CVB2, CVB3, CVB4, and CVB5.

In some cases, a method of the present disclosure can decrease the likelihood of an acute infection with CVB. In some cases, a method of the present disclosure can reduce the likelihood that an individual will develop a CVB infection-associated disease (e.g., CVB-induced gastrointestinal illness, myocarditis, pneumonia, aseptic meningitis, encephalitis, or hepatitis). In some cases, a method of the present disclosure can ameliorate one or more adverse symptoms of a CVB infection. In some cases, a method of the present disclosure treats one or more of CVB-induced gastrointestinal illness, myocarditis, pneumonia, aseptic meningitis, encephalitis, and hepatitis.

In some cases, where an individual to whom an immunogenic composition of the present disclosure is at greater risk than the general population for developing type 1 diabetes (T1D), a method of the present disclosure can reduce the likelihood that the individual will develop T1D.

In some cases, where an individual to whom an immunogenic composition of the present disclosure is at greater risk than the general population for developing celiac disease, a method of the present disclosure can reduce the likelihood that the individual will develop celiac disease.

### Subjects Suitable for Treatment

In some cases, an individual suitable for treatment according to the present disclosure is from 1 month to 3 months old (e.g., 1 month, 2 months, or 3 months old). In some cases, an individual suitable for treatment according to the present disclosure is from 3 months to 5 years of age. In some cases, an individual suitable for treatment according to the present disclosure is from 3 months to 6 months, from 6 months to 1 year, from 1 year to 2 years, or from 2 years to 5 years of age. In some cases, an individual suitable for treatment according to the present disclosure is from 5 years to 11 years of age. In some cases, an individual suitable for treatment according to the present disclosure is from 12 years to 17 years of age. In some cases, an individual suitable for treatment according to the present disclosure is from 18 years to 25 years of age. In some cases, an individual suitable for treatment according to the present disclosure is older than 25 years.

In some cases, the individual is greater than 6 years older, two years or older, six years or older, 12 years or older, 18 years or older, 30 years or older, between about 6 years and 12 years old, between about 6 years old and about 30 years old, between about 12 and about 30 years old, or between about 18 and about 30 years. In some cases, the individual is a pregnant woman.

In some cases, an individual suitable for treatment according to the present disclosure is immunologically naïve with respect to CVB; i.e., the individual has low or undetectable neutralizing antibody to CVB. In some cases, an individual suitable for treatment according to the present disclosure has previously been exposed (e.g., via natural infection) to CVB and has detectable antibody titer to CVB.

In some cases, the individual is at risk (e.g., at greater risk than the general population) of developing type 1 diabetes (T1D). Individuals at risk (e.g., at greater risk than the general population) of developing TID include individuals who have been genetically determined to be at increased risk for developing T1D, e.g., individuals with an HLA-conferred susceptibility to T1D, especially carriers of the HLA DR3 and/or DR4 alleles. Individuals at risk (e.g., at greater risk than the general population) of developing T1D include mothers or children with HLA-conferred disease susceptibility to T1D, especially carriers of the HLA DR3 and/or DR4 alleles; individuals with T1D in first or second-degree relatives; and individuals (e.g., children) testing positive for two or more diabetes-associated autoantibodies. In some cases, a subject method reduces the likelihood that an individual will develop T1D.

In some cases, the individual is at risk (e.g., at greater risk than the general population) of developing celiac disease. Such individuals include individuals have a genetic predisposition to developing celiac disease; and individuals who have tested positive for celiac-associated autoantibodies. In some cases, the individual is a carrier of an HLA-DR3 allele.

### EXAMPLES OF NON-LIMITING ASPECTS OF THE DISCLOSURE

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A composition comprising coxsackievirus B (CVB) of from two to five serotypes, wherein the CVB is the form of inactivated viral particles (IVP), viral-like particles (VLP), one or more CVB polypeptides, or one or more nucleic acids comprising nucleotide sequences encoding the one or more CVB polypeptides, wherein the composition comprises:
A) two or more of:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μL; or
B) two or more of:
a) CVB1 in an amount of from 1.0×10⁸ IVP to 2.5×10⁸ IVP;
b) CVB2 in an amount of from 5.0×10⁷ IVP to 1.5×10⁸ IVP;
c) CVB3 in an amount of from 5.0×10⁸ IVP to 1.5×10⁹ IVP;
d) CVB4 in an amount of from 1.0×10⁸ IVP to 2.5×10⁸ IVP; and
e) CVB5 in an amount of from 2.0×10⁸ IVP to 4.0×10⁸ IVP,
or an equivalent amount of VLP, one or more CVB polypeptides, or one or more nucleic acids comprising nucleotide sequences encoding the one or more CVB polypeptides,
in a volume of 500 μL,
wherein the composition does not include CVB6.

Aspect 2. The composition of aspect 1, wherein the composition comprises:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μl.

Aspect 3. The composition of aspect 2, wherein the composition comprises:
a) CVB1 in an amount of from 3.0×10⁷ IVP to 4.0×10⁷ IVP;
b) CVB2 in an amount of from 1.0×10⁷ IVP to 2.0×10⁷ IVP;
c) CVB3 in an amount of from 1.0×10⁸ IVP to 2.0×10⁸ IVP;
d) CVB4 in an amount of from 3.0×10⁷ IVP to 4.0×10⁷ IVP; and
e) CVB5 in an amount of from 6.0×10⁷ IVP to 6.0×10⁷ IVP,
or an equivalent amount of VLP, one or more CVB polypeptides, or one or more nucleic acids comprising nucleotide sequences encoding the one or more CVB polypeptides.

Aspect 4. A composition comprising coxsackievirus B (CVB) of from two to five serotypes, wherein the CVB is the form of inactivated viral particles (IVP), a CVB viral-like particle (VLP), one or more CVB polypeptides, or one or more nucleic acids comprising nucleotide sequences encoding the one or more CVB polypeptides wherein the composition, when administered to an individual, induces viral neutralizing antibody titer (VNT) of from 1/8 to 1/64,000 as determined by a VNT assay, wherein the composition does not include CVB6.

Aspect 5. The composition of aspect 4, wherein the composition comprises CVB of from two to five serotypes, wherein the CVB is the form of inactivated viral particles (IVP), wherein the composition comprises:
A) two or more of:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μL; or
B) two or more of:
a) CVB1 in an amount of from 1.0×10⁸ IVP to 2.5×10⁸ IVP;
b) CVB2 in an amount of from 5.0×10⁷ IVP to 1.5×10⁸ IVP;
c) CVB3 in an amount of from 5.0×10⁸ IVP to 1.5×10⁹ IVP;
d) CVB4 in an amount of from 1.0×10⁸ IVP to 2.5×10⁸ IVP; and
e) CVB5 in an amount of from 2.0×10⁸ IVP to 4.0×10⁸ IVP,
or an equivalent amount of VLP, one or more CVB polypeptides, or one or more nucleic acids comprising nucleotide sequences encoding the one or more CVB polypeptides,
in a volume of 500 μL.

Aspect 6. The composition of aspect 5, wherein the composition comprises:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides.

Aspect 7. The composition of aspect 6, wherein the composition comprises:
a) CVB1 in an amount of from 3.0×10⁷ IVP to 4.0×10⁷ IVP;
b) CVB2 in an amount of from 1.0×10⁷ IVP to 2.0×10⁷ IVP;
c) CVB3 in an amount of from 1.0×10⁸ IVP to 2.0×10⁸ IVP;
d) CVB4 in an amount of from 3.0×10⁷ IVP to 4.0×10⁷ IVP; and
e) CVB5 in an amount of from 6.0×10⁷ IVP to 6.0×10⁷ IVP,
or an equivalent amount of VLP, one or more CVB polypeptides, or one or more nucleic acids comprising nucleotide sequences encoding the one or more CVB polypeptides.

Aspect 8. The composition of any one of aspects 1-7, wherein the IVP are formalin inactivated.

Aspect 9. The composition of any one of aspects 1-8, wherein the composition comprises an adjuvant.

Aspect 10. The composition of any one of aspects 1-8, comprising saline.

Aspect 11. A method of inducing an immune response to coxsackievirus B (CVB) in an individual, the method comprising administering to the individual a composition of any one of aspects 1-10.

Aspect 12. The method of aspect 11, wherein the composition is administered intramuscularly.

Aspect 13. The method of aspect 11, wherein the composition is administered subcutaneously.

Aspect 14. A method of reducing the likelihood that an individual will develop an acute coxsackievirus B (CVB) infection, the method comprising administering to the individual a composition of any one of aspects 1-10.

Aspect 15. The method of any one of aspects 11-14, wherein the individual is a neonate.

Aspect 16. The method of any one of aspects 11-14, wherein the individual is a pregnant woman.

Aspect 17. The method of any one of aspects 11-14, wherein the individual is at increased risk of developing type 1 diabetes.

Aspect 18. The method of any one of aspects 11-14, wherein the individual is at increased risk of developing celiac disease.

Aspect 19. The method of any one of aspects 11-14, wherein the individual is a carrier of an HLA DR3 and/or an HLA DR4 allele.

Aspect 20. The method of any one of aspects 11-14, wherein the individual was seropositive for viral neutralizing titer to one or more of CVB1, CVB2, CVB3, CVB4, and CVB5 prior to said administering.

Aspect 21. The method of any one of aspects 11-14, wherein the individual was seronegative for viral neutralizing titer to one or more of CVB1, CVB2, CVB3, CVB4, and CVB5 prior to said administering.

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A composition comprising coxsackievirus B (CVB) of from two to five serotypes, wherein the CVB is the form of inactivated viral particles (IVP), viral-like particles (VLP), one or more CVB polypeptides, or one or more nucleic acids comprising nucleotide sequences encoding the one or more CVB polypeptides, wherein the composition comprises:
A) two or more of:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μL; or
B) two or more of:
a) CVB1 in an amount of from 1.0×10⁸ IVP to 2.5×10⁸ IVP;
b) CVB2 in an amount of from 5.0×10⁷ IVP to 1.5×10⁸ IVP;
c) CVB3 in an amount of from 5.0×10⁸ IVP to 1.5×10⁹ IVP;
d) CVB4 in an amount of from 1.0×10⁸ IVP to 2.5×10⁸ IVP; and
e) CVB5 in an amount of from 2.0×10⁸ IVP to 4.0×10⁸ IVP,
or an equivalent amount of VLP, one or more CVB polypeptides, or one or more nucleic acids comprising nucleotide sequences encoding the one or more CVB polypeptides,
in a volume of 500 μL; or
C) two or more of:
a) CVB1 protein in an amount of from 1 μg to 50 μg;
b) CVB2 protein in an amount of from 1 μg to 50 μg;
c) CVB3 protein in an amount of from 1 μg to 50 μg;
d) CVB4 protein in an amount of from 1 μg to 50 μg; and
e) CVB5 protein in an amount of from 1 μg to 50 μg,
wherein the composition does not include CVB6.

Aspect 2. The composition of aspect 1, wherein the composition comprises:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μl.

Aspect 3. The composition of aspect 2, wherein the composition comprises:
a) CVB1 in an amount of from 3.0×10⁷ IVP to 4.0×10⁷ IVP;
b) CVB2 in an amount of from 1.0×10⁷ IVP to 2.0×10⁷ IVP;
c) CVB3 in an amount of from 1.0×10⁸ IVP to 2.0×10⁸ IVP;
d) CVB4 in an amount of from 3.0×10⁷ IVP to 4.0×10⁷ IVP; and
e) CVB5 in an amount of from 6.0×10⁷ IVP to 6.0×10⁷ IVP,
or an equivalent amount of VLP, one or more CVB polypeptides, or one or more nucleic acids comprising nucleotide sequences encoding the one or more CVB polypeptides.

Aspect 4. The composition of aspect 1, wherein the composition comprises:
a) CVB1 in an amount of 3.3×10⁷ IVP;
b) CVB2 in an amount of 1.1×10⁷ IVP;
c) CVB3 in an amount of 1.5×10⁸ IVP;
d) CVB4 in an amount of 3.2×10⁷ IVP; and
e) CVB5 in an amount of 6.3×10⁷ IVP,
or an equivalent amount of VLP, one or more CVB polypeptides, or one or more nucleic acids comprising nucleotide sequences encoding the one or more CVB polypeptides,
in a volume of 100 μl.

Aspect 5. The composition of aspect 1, wherein the composition comprises:
a) CVB1 in an amount of 1.6×10⁸ IVP;
b) CVB2 in an amount of 5.5×10⁷ IVP;
c) CVB3 in an amount of 7.5×10⁸ IVP;
d) CVB4 in an amount of 1.6×10⁸ IVP; and
e) CVB5 in an amount of 3.1×10⁸ IVP,
or an equivalent amount of VLP, one or more CVB polypeptides, or one or more nucleic acids comprising nucleotide sequences encoding the one or more CVB polypeptides,
in a volume of 500 μL.

Aspect 6. The composition of aspect 1, wherein the composition comprises:
a) CVB1 protein in an amount of 2.8 μg;
b) CVB2 protein in an amount of 1.7 μg;
c) CVB3 protein in an amount of 2.7 μg;
d) CVB4 protein in an amount of 1.2 μg; and
e) CVB5 protein in an amount of 2.3 μg,
in a volume of 100 μl.

Aspect 7. The composition of aspect 1, wherein the composition comprises:
a) CVB1 protein in an amount of 14 μg;
b) CVB2 protein in an amount of 8.5 μg;
c) CVB3 protein in an amount of 13.5 μg;
d) CVB4 protein in an amount of 6.0 μg; and
e) CVB5 protein in an amount of 11.5 μg,
in a volume of 500 μl.

Aspect 8. A composition comprising coxsackievirus B (CVB) of from two to five serotypes, wherein the CVB is the form of inactivated viral particles (IVP), a CVB viral-like particle (VLP), one or more CVB polypeptides, or one or more nucleic acids comprising nucleotide sequences encoding the one or more CVB polypeptides wherein the composition, when administered to an individual, induces viral neutralizing antibody titer (VNT) of from 1/8 to 1/64,000 as determined by a VNT assay, wherein the composition does not include CVB6.

Aspect 9. The composition of aspect 8, wherein the composition comprises CVB of from two to five serotypes, wherein the CVB is the form of inactivated viral particles (IVP), wherein the composition comprises:
A) two or more of:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μL; or
B) two or more of:
a) CVB1 in an amount of from 1.0×10⁸ IVP to 2.5×10⁸ IVP;
b) CVB2 in an amount of from 5.0×10⁷ IVP to 1.5×10⁸ IVP;
c) CVB3 in an amount of from 5.0×10⁸ IVP to 1.5×10⁹ IVP;
d) CVB4 in an amount of from 1.0×10⁸ IVP to 2.5×10⁸ IVP; and
e) CVB5 in an amount of from 2.0×10⁸ IVP to 4.0×10⁸ IVP; or
C) two or more of:
a) CVB1 protein in an amount of from 1 μg to 50 μg;
b) CVB2 protein in an amount of from 1 μg to 50 μg;
c) CVB3 protein in an amount of from 1 μg to 50 μg;
d) CVB4 protein in an amount of from 1 μg to 50 μg; and
e) CVB5 protein in an amount of from 1 μg to 50 μg,
or an equivalent amount of VLP, one or more CVB polypeptides, or one or more nucleic acids comprising nucleotide sequences encoding the one or more CVB polypeptides,
in a volume of 500 μL.

Aspect 10. The composition of aspect 9, wherein the composition comprises:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides.

Aspect 11. The composition of aspect 10, wherein the composition comprises:
a) CVB1 in an amount of from 3.0×10⁷ IVP to 4.0×10⁷ IVP;
b) CVB2 in an amount of from 1.0×10⁷ IVP to 2.0×10⁷ IVP;
c) CVB3 in an amount of from 1.0×10⁸ IVP to 2.0×10⁸ IVP;
d) CVB4 in an amount of from 3.0×10⁷ IVP to 4.0×10⁷ IVP; and
e) CVB5 in an amount of from 6.0×10⁷ IVP to 6.0×10⁷ IVP,
or an equivalent amount of VLP, one or more CVB polypeptides, or one or more nucleic acids comprising nucleotide sequences encoding the one or more CVB polypeptides.

Aspect 12. The composition of aspect 9, wherein the composition comprises:
a) CVB1 in an amount of 3.3×10⁷ IVP;
b) CVB2 in an amount of 1.1×10⁷ IVP;
c) CVB3 in an amount of 1.5×10⁸ IVP;
d) CVB4 in an amount of 3.2×10⁷ IVP; and
e) CVB5 in an amount of 6.3×10⁷ IVP,
or an equivalent amount of VLP, one or more CVB polypeptides, or one or more nucleic acids comprising nucleotide sequences encoding the one or more CVB polypeptides,
in a volume of 100 μl.

Aspect 13. The composition of aspect 9, wherein the composition comprises:
a) CVB1 in an amount of 1.6×10⁸ IVP;
b) CVB2 in an amount of 5.5×10⁷ IVP;
c) CVB3 in an amount of 7.5×10⁸ IVP;
d) CVB4 in an amount of 1.6×10⁸ IVP; and
e) CVB5 in an amount of 3.1×10⁸ IVP,
or an equivalent amount of VLP, one or more CVB polypeptides, or one or more nucleic acids comprising nucleotide sequences encoding the one or more CVB polypeptides,
in a volume of 500 μL.

Aspect 14. The composition of aspect 9, wherein the composition comprises:
a) CVB1 protein in an amount of 2.8 μg;
b) CVB2 protein in an amount of 1.7 μg;
c) CVB3 protein in an amount of 2.7 μg;
d) CVB4 protein in an amount of 1.2 μg; and
e) CVB5 protein in an amount of 2.3 μg,
in a volume of 100 μl.

Aspect 15. The composition of aspect 9, wherein the composition comprises:
a) CVB1 protein in an amount of 14 μg;
b) CVB2 protein in an amount of 8.5 μg;
c) CVB3 protein in an amount of 13.5 μg;
d) CVB4 protein in an amount of 6.0 μg; and
e) CVB5 protein in an amount of 11.5 μg,
in a volume of 500 μl.

Aspect 16. The composition of any one of aspects 1-5 and 8-13, wherein the IVP are formalin inactivated.

Aspect 17. The composition of any one of aspects 1-16, wherein the composition comprises an adjuvant.

Aspect 18. The composition of any one of aspects 1-17, comprising saline.

Aspect 19. A method of inducing an immune response to coxsackievirus B (CVB) in an individual, the method comprising administering to the individual a composition of any one of aspects 1-18.

Aspect 20. The method of aspect 19, wherein the composition is administered intramuscularly.

Aspect 21. The method of aspect 19, wherein the composition is administered subcutaneously.

Aspect 22. A method of reducing the likelihood that an individual will develop an acute coxsackievirus B (CVB) infection or a CVB infection-associated disease, the method comprising administering to the individual a composition of any one of aspects 1-18.

Aspect 23. The method of any one of aspects 19-22, wherein the individual is a neonate.

Aspect 24. The method of any one of aspects 19-22, wherein the individual is a pregnant woman.

Aspect 25. The method of any one of aspects 19-22, wherein the individual is at increased risk of developing type 1 diabetes.

Aspect 26. The method of any one of aspects 19-22, wherein the individual is at increased risk of developing celiac disease.

Aspect 27. The method of any one of aspects 19-22, wherein the individual is a carrier of an HLA DR3 and/or an HLA DR4 allele.

Aspect 28. The method of any one of aspects 19-22, wherein the individual was seropositive for viral neutralizing titer to one or more of CVB1, CVB2, CVB3, CVB4, and CVB5 prior to said administering.

Aspect 29. The method of any one of aspects 19-22, wherein the individual was seronegative for viral neutralizing titer to one or more of CVB1, CVB2, CVB3, CVB4, and CVB5 prior to said administering.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Induction of VNT in Human Subjects

A composition comprising CVB of serotypes 1-5 was administered to normal, healthy human volunteers. Two cohorts were randomized in a 3 to 1 fashion to receive either 100 μL or 500 μL of the composition, or a matching placebo. Subjects received 3 doses at monthly intervals. Analysis of VNT was conducted after all subjects completed 3 doses, and 1 month of follow up after the last dose. The composition is a formalin-inactivated vaccine comprising CVB serotypes 1 through 5 in the form of inactivated viral particles (IVP). IVP is equivalent to RNA copy number.

The 100 μL (“low dose”) composition included CVB1-CVB5 in the following amounts:
a) CVB1: 3.3×10⁷ IVP;
b) CVB2: 1.1×10⁷ IVP;
c) CVB3 1.5×10⁸ IVP;
d) CVB4: 3.2×10⁷ IVP; and
e) CVB5: 6.3×10⁷ IVP.

The 500 μL (“high dose”) composition included CVB1-CVB5 in the following amounts:
a) CVB1: 1.6×10⁸ IVP;
b) CVB2: 5.5×10⁷ IVP;
c) CVB3: 7.5×10⁸ IVP;
d) CVB4: 1.6×10⁸ IVP; and
e) CVB5: 3.1×10⁸ IVP.

No treatment-emergent serious adverse events (SAEs) were observed in any of the subjects. No treatment-emergent adverse events of special interest (AESI) were observed in any of the subjects.

Dose-dependent generation of high virus neutralizing titers (VNT) were observed. Blue: low dose; red: high dose. The data are shown in FIG. 2. As shown in FIG. 2, titers in the range of 1/2,000-1/4,000 were observed. Pre-existing anti-CVB viral titers in ˜40% of subjects explain baseline VNT in all groups. Peak VNT titer of antibodies for each serotype are shown in FIG. 3.

### VNT in Seropositive and Seronegative Individuals

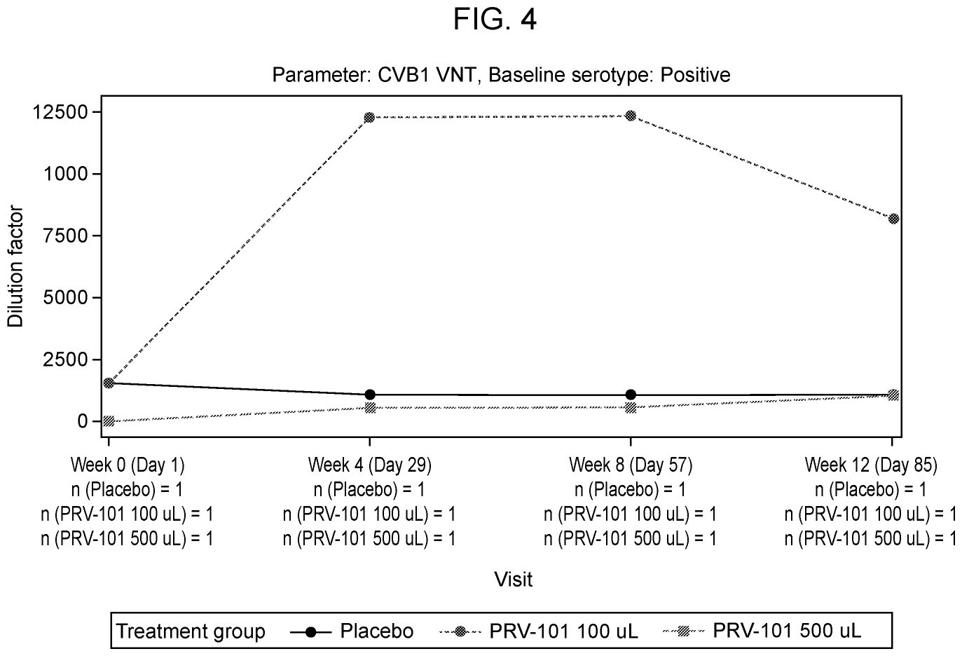

VNT to each of CVB1 through CVB5 was tested both pre-vaccination and post-vaccination. The data are depicted in FIG. 4-13. FIG. 4-13 provide mean (+/−standard deviation) curves of anti-CVB virus neutralizing antibody titer by visit, baseline seroconversion and serotype.

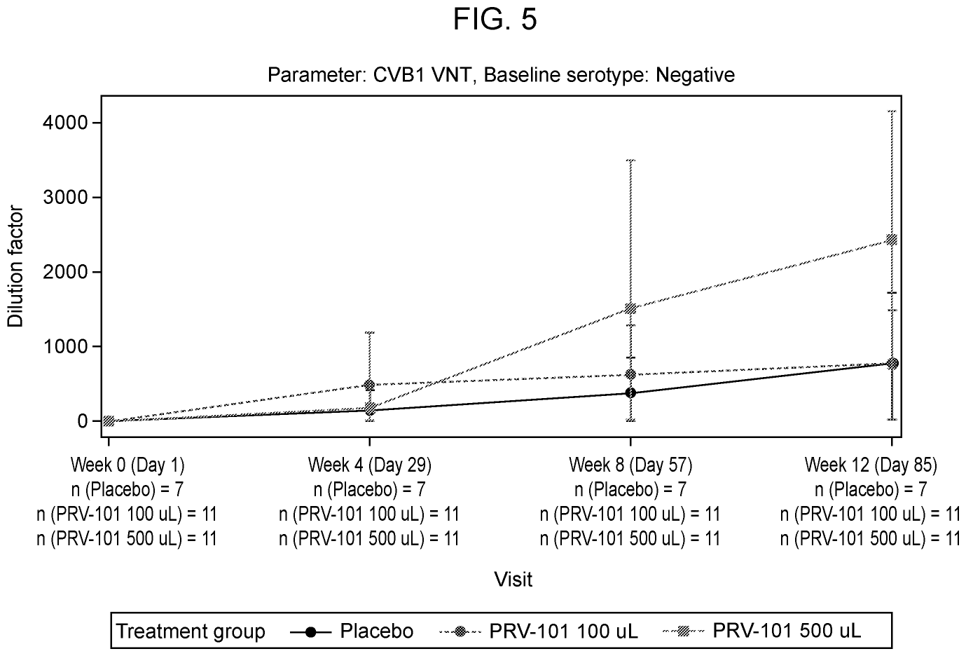

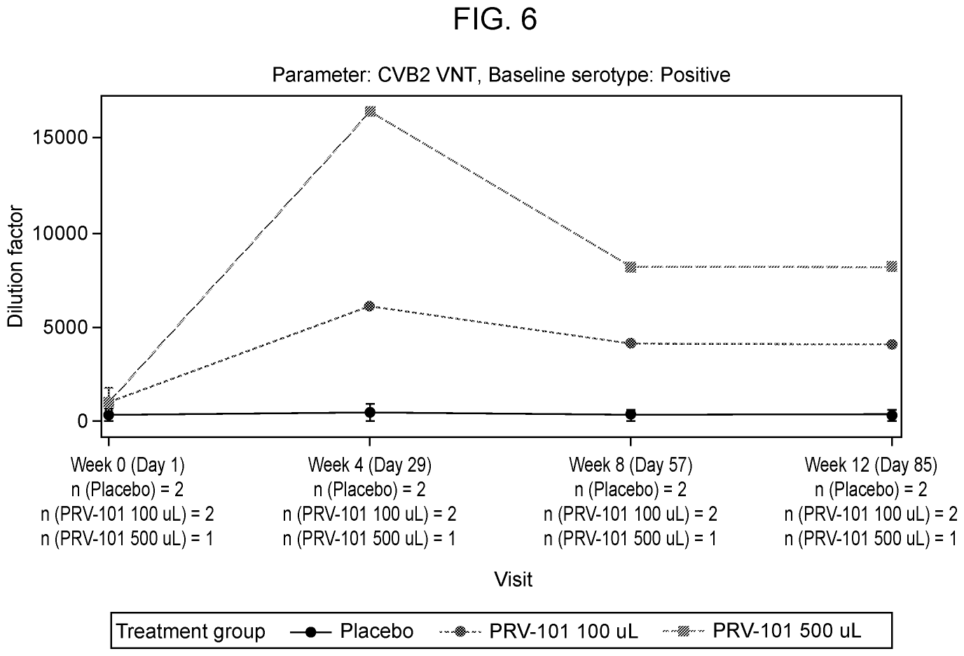

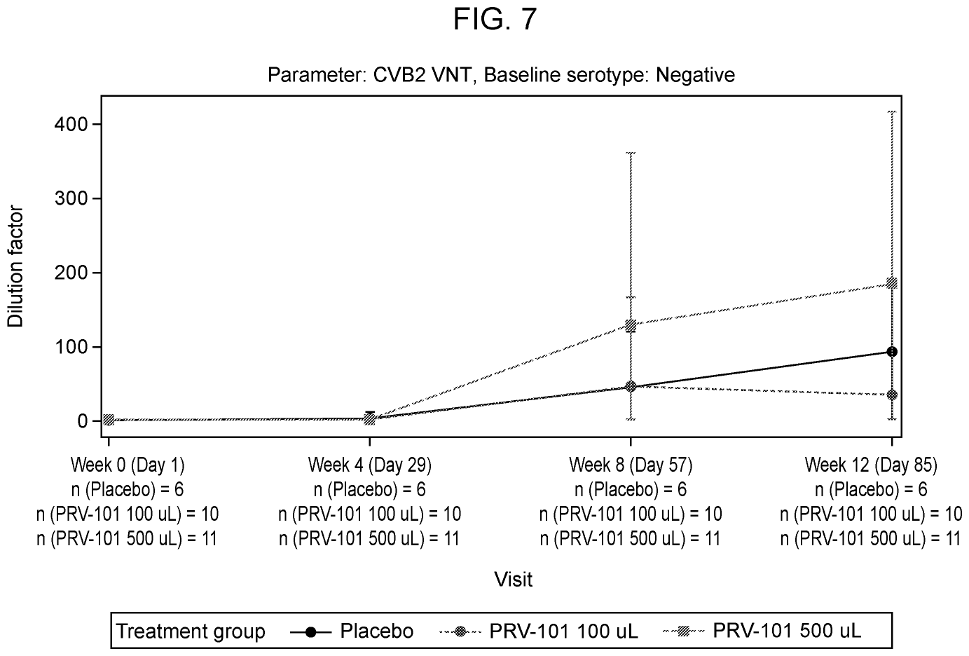

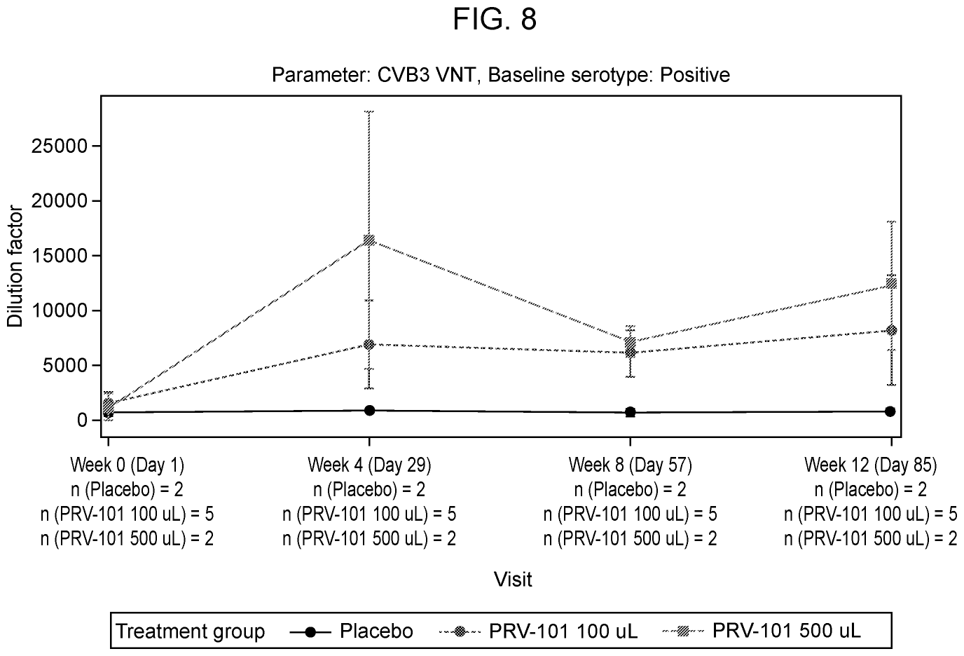

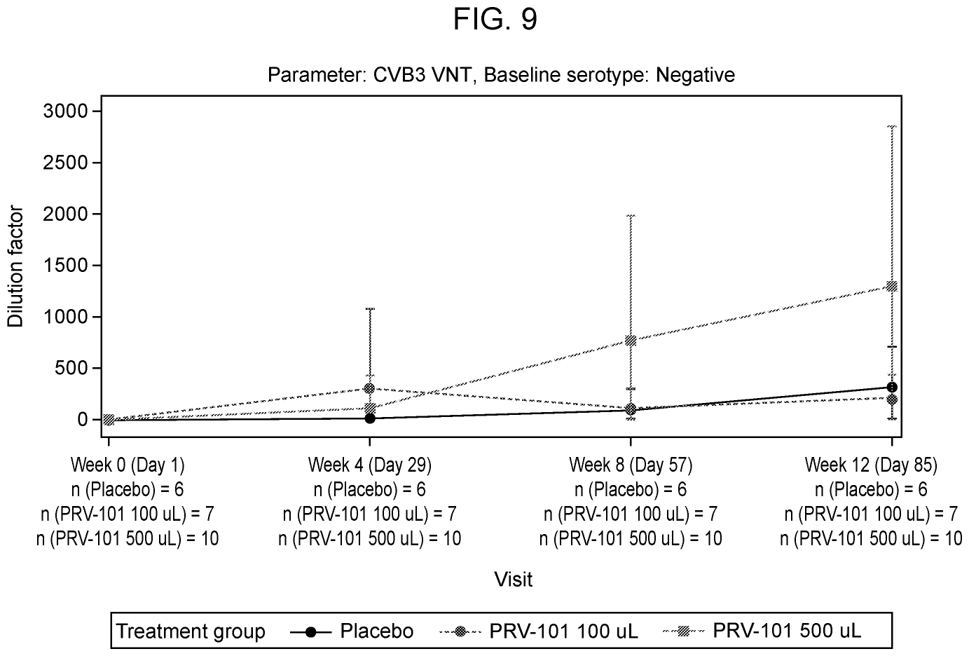

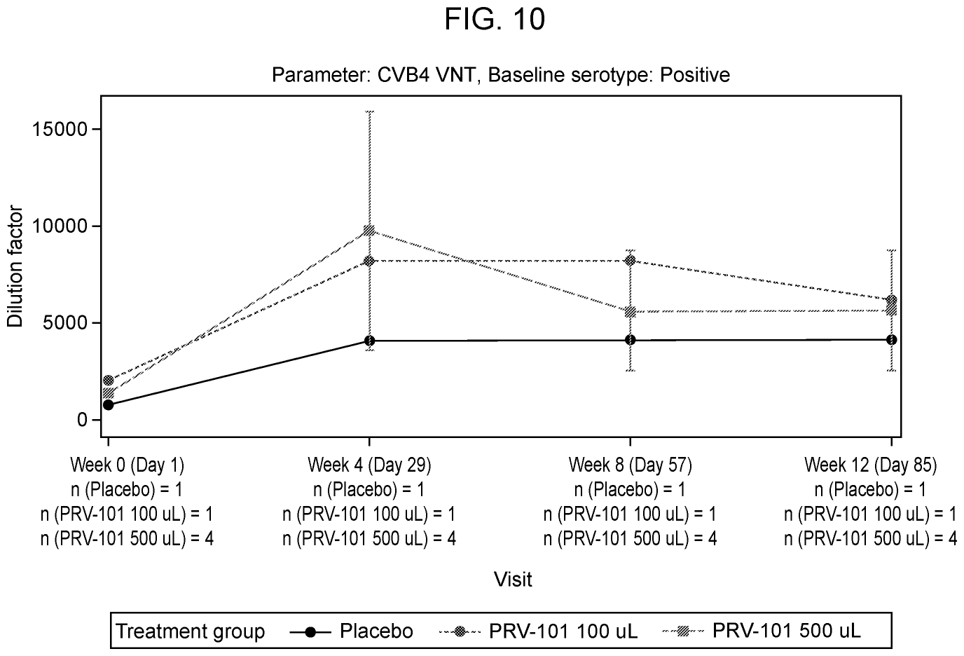

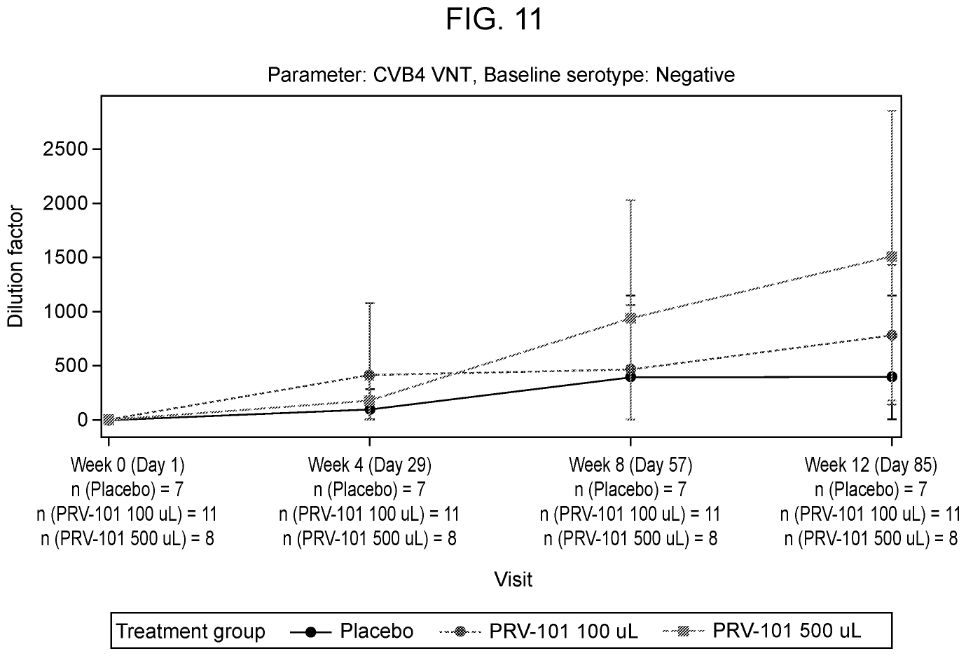

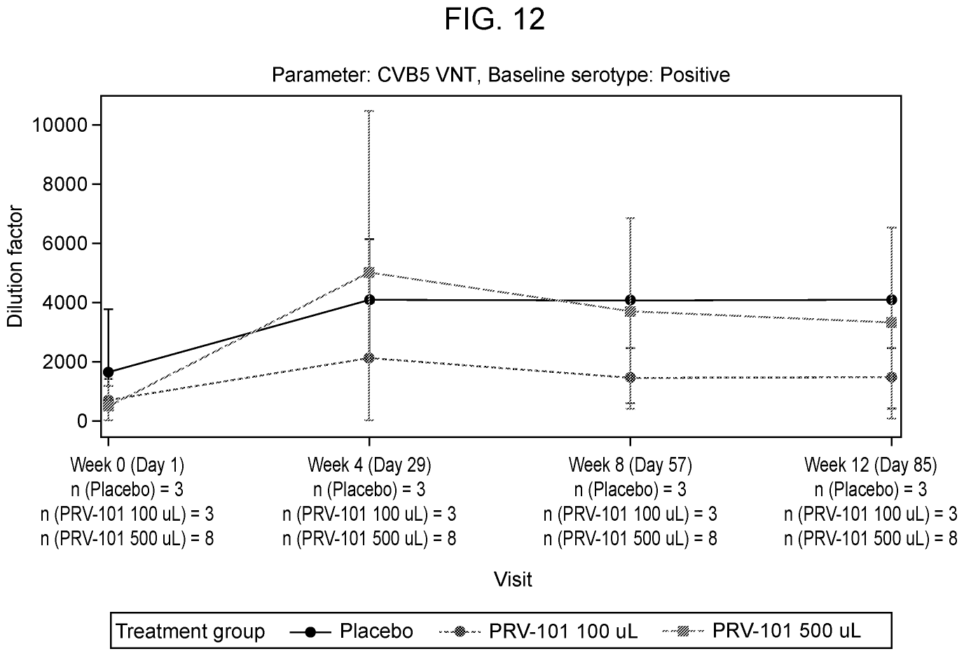

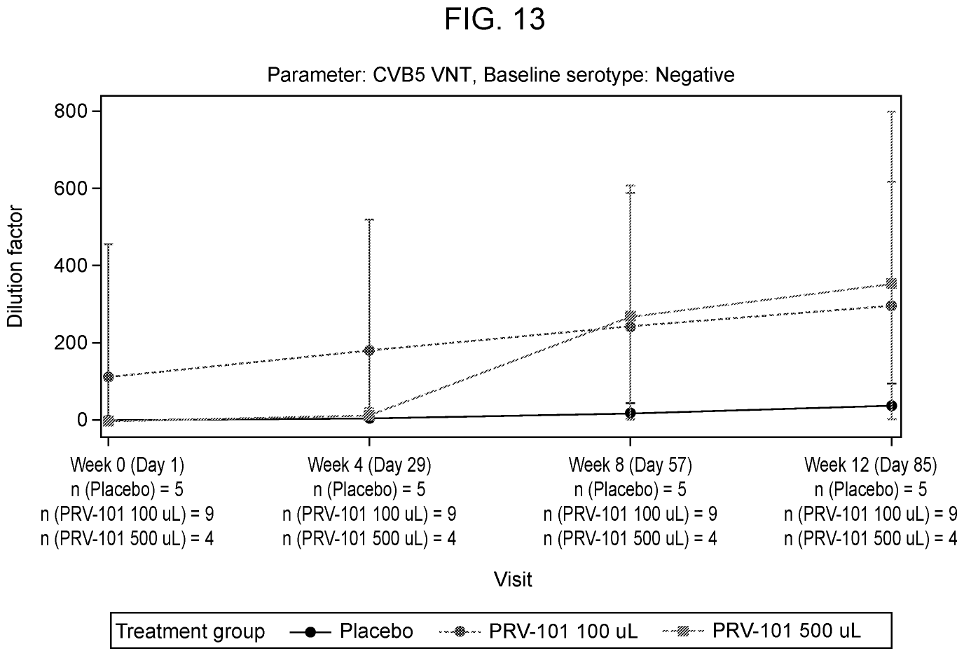

FIG. 4-13 depict VNT to CVB1 (FIG. 4 and FIG. 5), CVB2 (FIG. 6 and FIG. 7), CVB3 (FIG. 8 and FIG. 9), CVB4 (FIG. 10 and FIG. 11), and CBV5 (FIG. 12 and FIG. 13) in serum of vaccinated human subjects. The subjects were either seropositive for VNT to the indicated CVB serotype before vaccination (FIGS. 4, 6, 8, 10, and 12) or seronegative before vaccination (FIGS. 5, 7, 9, 11, and 13).

### Response Rates

The response rate of the vaccinated human subjects was assessed. Responders were defined as either: a) subjects who are seronegative at baseline and who seroconvert (i.e., VNT≥1/4) at any post-vaccination time point; or b) subjects who are positive for antibodies at baseline (i.e., subjects who have pre-existing antibodies) and have a 4-fold or greater increase in VNT at any post-vaccination time point.

The data are shown in Table 1, below.

TABLE 1

| | Placebo | Low Dose | High Dose |
|---|---|---|---|
| CVB1 | 12.5 | 100 | 100 |
| CVB2 | 0 | 100 | 100 |
| CVB3 | 0 | 75 | 100 |
| CVB4 | 0 | 100 | 100 |
| CVB5 | 0 | 91.7 | 100 |
| All of CVB1-CVB5 | 0 | 66.7 | 100 |

### Results at 6-Month Time Point (Week 32)

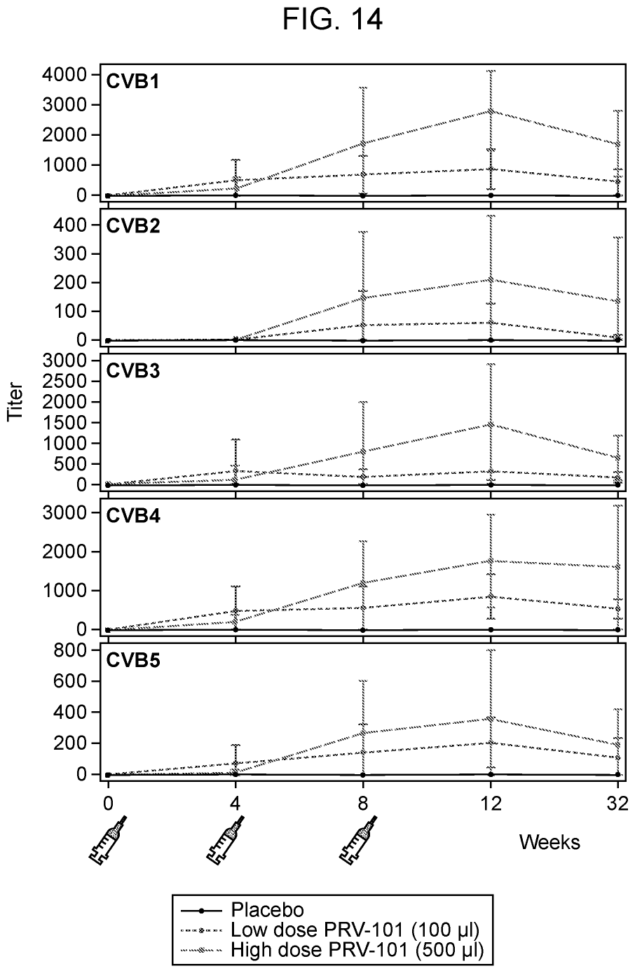

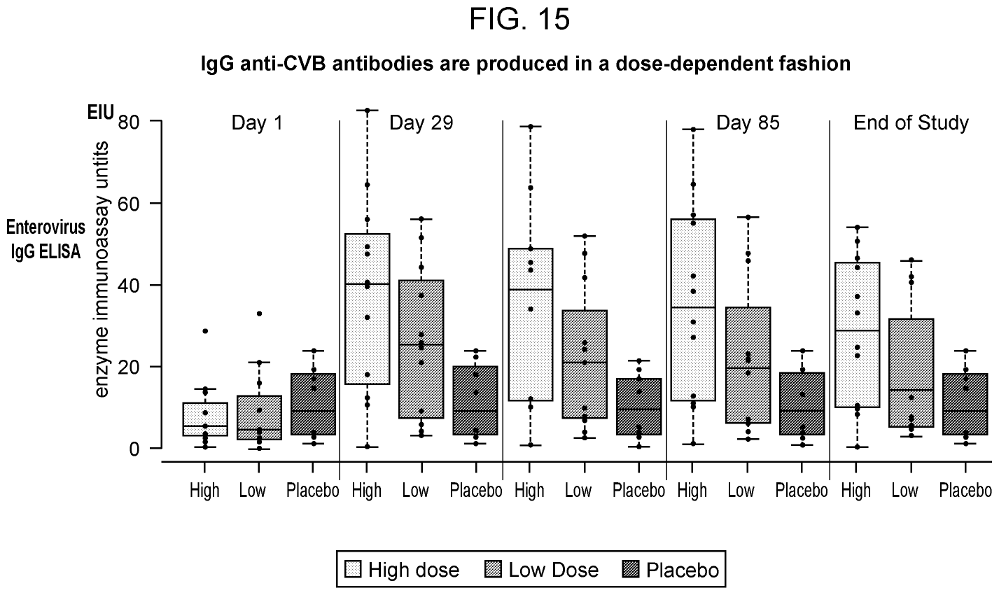

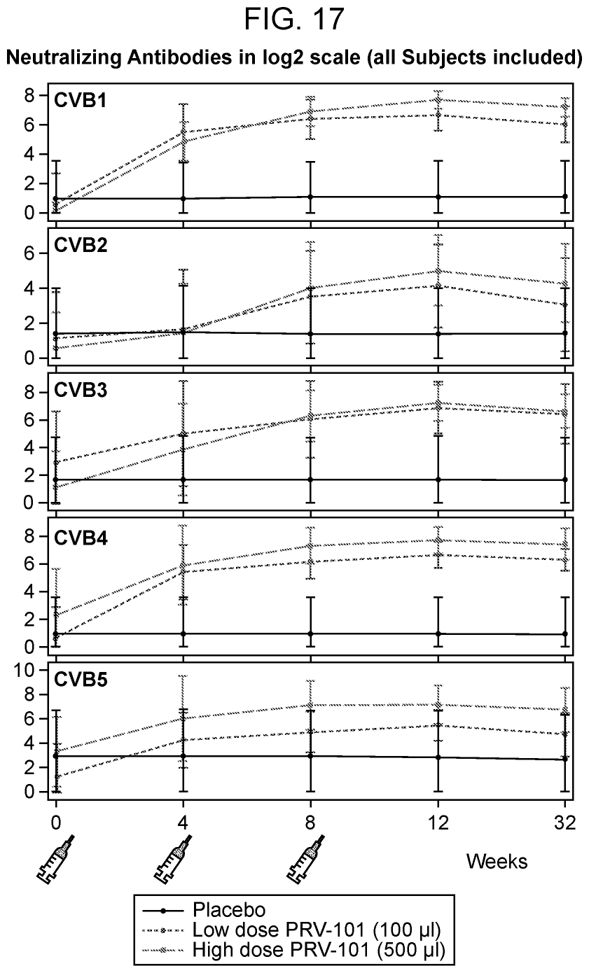

The level of VNT was assayed at various time points (weeks 4, 8, 12, and 32) after administration of the first dose of the CVB vaccine composition. The results are shown in FIG. 14, FIG. 15, and FIG. 17.

As shown in FIG. 14, of the baseline seronegative subjects, at week 32 (i.e., 24 weeks after the last dose on week 12), 90% in the high-dose group had titers (VNT) equal of greater than 1/8 against all 5 serotypes. As shown in FIG. 14, at week 32, 75% of subjects in the low-dose group had titers equal or greater than 1/8 against all 5 serotypes.

As shown in FIG. 15, a commercial ELISA assay (DRG International) showed dose- and time-dependent durable enterovirus-specific IgG responses.

FIG. 17 depicts the CVB-neutralizing antibody titer in all subjects (baseline seropositive and baseline seronegative). The titers are presented in a logarithmic (log 2) scale. In addition to the time- and dose-response, the durability of the response at Week 32 is apparent for all serotypes in the vaccine.

The data show durability of VNT responses against all strains of CVB in the vaccine (CVB1-5). At this 6-month post-last administration final time point (Week 32), the percent of subjects in the high dose PRV-01 arm who maintained high titers of VNT against 4 serotypes was 100%, and 90% for all 5 serotypes included in the vaccine. These data confirmed the durability of the response. High titers are defined as >=1/8, a level which has been seen to be clinically protective in the only other enteroviral vaccine on the market, the inactivated poliovirus vaccine. Evidence of VNT cross-reactivity was observed against CVB6, the less common CVB serotype not included in the CVB vaccine composition. In addition to VNT, commercial ELISA assays showed dose- and time-dependent durable IgG responses after the switch from initial robust IgM responses, as well as partial IgA responses (which indicate a degree of mucosal immunity).

### Example 2

A composition comprising CVB of serotypes 1-5 was administered to normal, healthy human volunteers. Two cohorts were randomized in a 3 to 1 fashion to receive either 100 μL or 500 μL of the composition, or a matching placebo. Subjects received 3 doses at monthly intervals. Analysis of VNT was conducted after all subjects completed 3 doses. A final analysis was conducted 6 months following the last dose of the vaccine (week 32 of the study). The composition is a formalin-inactivated vaccine comprising CVB serotypes 1 through 5 in the form of IVP.

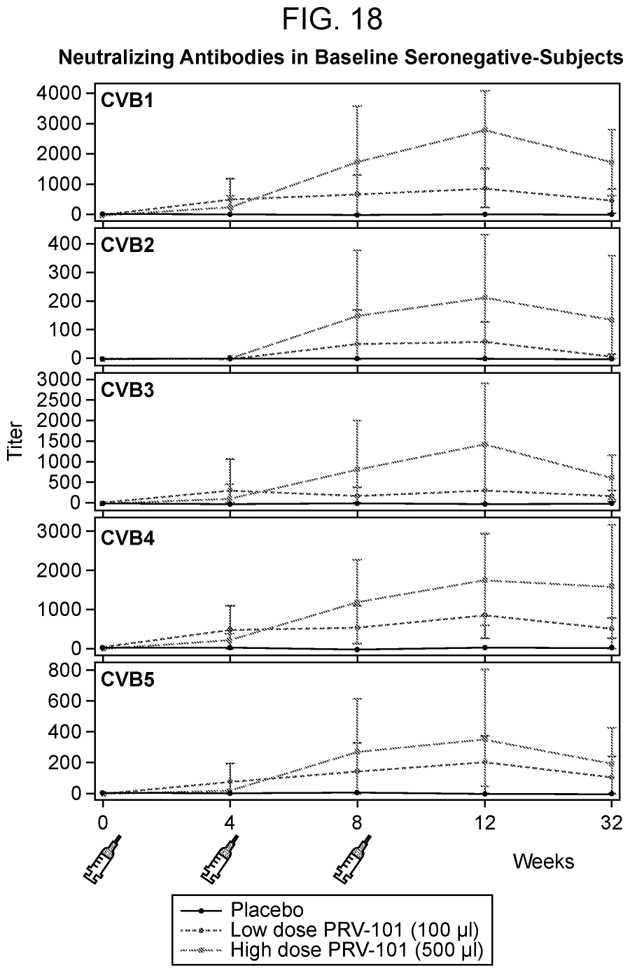

The results indicate that high titers of VNT against all 5 serotypes were induced, in a dose-dependent fashion. The response is durable: 6 months after the last dose of vaccine, high-level protective antibodies were achieved in 100% of subjects for the majority of serotypes included in the vaccine, and no less than 90% for all. The data are shown in FIG. 17 and FIG. 18.

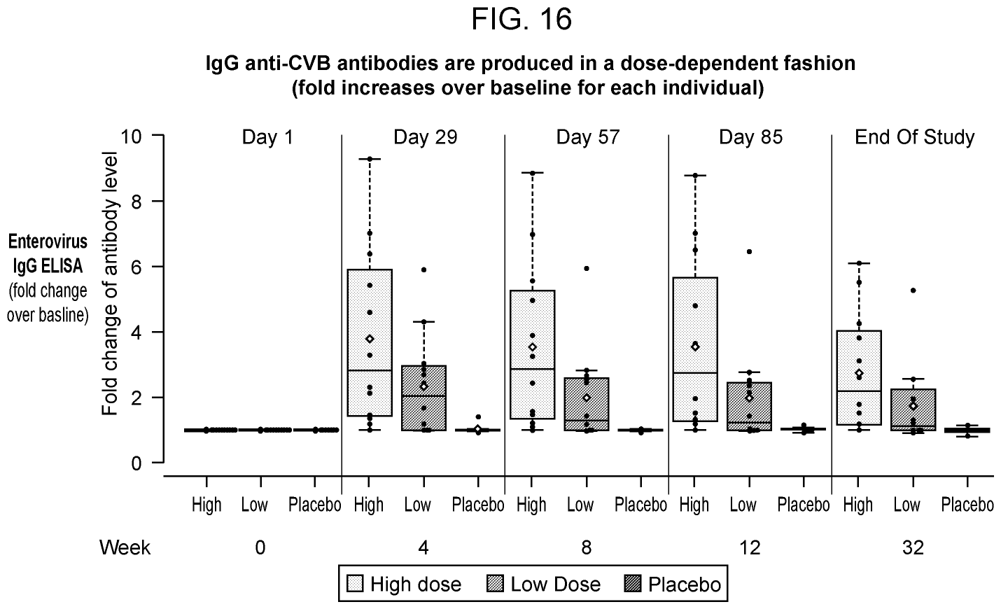

As shown in FIG. 16, IgG anti-CVB antibodies were produced in a dose-dependent fashion. The data demonstrate the durability of the IgG response at week 32. Similar data were obtained for IgM.

FIG. 18 shows dose-dependent generation of high titers of VNT for all serotypes (CVB1, CVB2, CVB3, CVB4, and CVB5). Of the baseline seronegative subjects, at week 32, 100% of the subjects had neutralizing antibody titers of 1/8 or more against at least 4 serotypes, and no less than 90% of the subjects had neutralizing antibody titers of 1/8 or more against all 5 serotypes included in the vaccine.

### Example 3

Neutralizing antibodies were measured against CVB6 and Coxsackievirus A9 (CAV9), using a plaque neutralization antibody assay. Neutralizing antibodies were analyzed from the baseline serum sample taken at the time of the first vaccination as well as from samples taken a month after the 3rd vaccination.

Altogether, 64 serum samples were analyzed for neutralizing CVB antibodies including 32 V2, and 32 VII samples. Antibody levels in these two time-points indicate whether the PRV-101 vaccine induces cross-reactive antibodies in initially CVB6/CAV9 seronegative subjects or increases antibody titers in initially seropositive subjects. The results showed that PRV-101 vaccine induced a certain degree of cross-reactive neutralizing antibodies against CVB6. No cross reactivity was observed against CAV9.

Altogether 8 trial participants developed seroconversion or 4-fold or higher increase in neutralizing antibodies against CVB6. All 8 responders belonged to the vaccinated trial arms and none belonged to the placebo arm. None of the trial participants developed response to CVA9. The data indicate that, given the similarity between CVB viruses, vaccines that contains some but not all six known CVB serotypes could raise protection against all of six CVB serotypes. An immunogenic composition therefore could be a vaccine that is based in 1 to 5 CVB strains but that raises protection against one or more of the CVB strains that were not on the vaccine.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A composition comprising coxsackievirus B (CVB) of from two to five serotypes, wherein the CVB is the form of inactivated viral particles (IVP), viral-like particles (VLP), one or more CVB polypeptides, or one or more nucleic acids comprising nucleotide sequences encoding the one or more CVB polypeptides, wherein the composition comprises:
A) two or more of:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μL; or
B) two or more of:
a) CVB1 in an amount of from 1.0×10⁸ IVP to 2.5×10⁸ IVP;
b) CVB2 in an amount of from 5.0×10⁷ IVP to 1.5×10⁸ IVP;
c) CVB3 in an amount of from 5.0×10⁸ IVP to 1.5×10⁹ IVP;
d) CVB4 in an amount of from 1.0×10⁸ IVP to 2.5×10⁸ IVP; and
e) CVB5 in an amount of from 2.0×10⁸ IVP to 4.0×10⁸ IVP,
or an equivalent amount of VLP, one or more CVB polypeptides, or one or more nucleic acids comprising nucleotide sequences encoding the one or more CVB polypeptides,
in a volume of 500 μL; or
C) two or more of:
a) CVB1 protein in an amount of from 1 μg to 50 μg;
b) CVB2 protein in an amount of from 1 μg to 50 μg;
c) CVB3 protein in an amount of from 1 μg to 50 μg;
d) CVB4 protein in an amount of from 1 μg to 50 μg; and
e) CVB5 protein in an amount of from 1 μg to 50 μg,
wherein the composition does not include CVB6.

2. The composition of claim 1, wherein the composition comprises:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μl; or
wherein the composition comprises:
a) CVB1 in an amount of from 3.0×10⁷ IVP to 4.0×10⁷ IVP;
b) CVB2 in an amount of from 1.0×10⁷ IVP to 2.0×10⁷ IVP;
c) CVB3 in an amount of from 1.0×10⁸ IVP to 2.0×10⁸ IVP;
d) CVB4 in an amount of from 3.0×10⁷ IVP to 4.0×10⁷ IVP; and
e) CVB5 in an amount of from 6.0×10⁷ IVP to 6.0×10⁷ IVP,
or an equivalent amount of VLP, one or more CVB polypeptides, or one or more nucleic acids comprising nucleotide sequences encoding the one or more CVB polypeptides,
in a volume of 100 μl; or
wherein the composition comprises:
a) CVB1 in an amount of 3.3×10⁷ IVP;
b) CVB2 in an amount of 1.1×10⁷ IVP;
c) CVB3 in an amount of 1.5×10⁸ IVP;
d) CVB4 in an amount of 3.2×10⁷ IVP; and
e) CVB5 in an amount of 6.3×10⁷ IVP,
or an equivalent amount of VLP, one or more CVB polypeptides, or one or more nucleic acids comprising nucleotide sequences encoding the one or more CVB polypeptides,
in a volume of 100 μl; or
wherein the composition comprises:
a) CVB1 in an amount of 1.6×10⁸ IVP;
b) CVB2 in an amount of 5.5×10⁷ IVP;
c) CVB3 in an amount of 7.5×10⁸ IVP;
d) CVB4 in an amount of 1.6×10⁸ IVP; and
e) CVB5 in an amount of 3.1×10⁸ IVP,
or an equivalent amount of VLP, one or more CVB polypeptides, or one or more nucleic acids comprising nucleotide sequences encoding the one or more CVB polypeptides,
in a volume of 500 μL.

3.-5. (canceled)

6. The composition of claim 1, wherein the composition comprises:
a) CVB1 protein in an amount of 2.8 μg;
b) CVB2 protein in an amount of 1.7 μg;
c) CVB3 protein in an amount of 2.7 μg;
d) CVB4 protein in an amount of 1.2 μg; and
e) CVB5 protein in an amount of 2.3 μg,
in a volume of 100 μl; or
wherein the composition comprises:
a) CVB1 protein in an amount of 14 μg;
b) CVB2 protein in an amount of 8.5 μg;
c) CVB3 protein in an amount of 13.5 μg;
d) CVB4 protein in an amount of 6.0 μg; and
e) CVB5 protein in an amount of 11.5 μg,
in a volume of 500 μl.

7. (canceled)

8. A composition comprising coxsackievirus B (CVB) of from two to five serotypes, wherein the CVB is the form of inactivated viral particles (IVP), a CVB viral-like particle (VLP), one or more CVB polypeptides, or one or more nucleic acids comprising nucleotide sequences encoding the one or more CVB polypeptides wherein the composition, when administered to an individual, induces viral neutralizing antibody titer (VNT) of from 1/8 to 1/64,000 as determined by a VNT assay, wherein the composition does not include CVB6.

9. The composition of claim 8, wherein the composition comprises CVB of from two to five serotypes, wherein the CVB is the form of inactivated viral particles (IVP), wherein the composition comprises:
A) two or more of:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides,
in a volume of 100 μL; or
B) two or more of:
a) CVB1 in an amount of from 1.0×10⁸ IVP to 2.5×10⁸ IVP;
b) CVB2 in an amount of from 5.0×10⁷ IVP to 1.5×10⁸ IVP;
c) CVB3 in an amount of from 5.0×10⁸ IVP to 1.5×10⁹ IVP;
d) CVB4 in an amount of from 1.0×10⁸ IVP to 2.5×10⁸ IVP; and
e) CVB5 in an amount of from 2.0×10⁸ IVP to 4.0×10⁸ IVP; or
C) two or more of:
a) CVB1 protein in an amount of from 1 μg to 50 μg;
b) CVB2 protein in an amount of from 1 μg to 50 μg;
c) CVB3 protein in an amount of from 1 μg to 50 μg;
d) CVB4 protein in an amount of from 1 μg to 50 μg; and
e) CVB5 protein in an amount of from 1 μg to 50 μg,
or an equivalent amount of VLP, one or more CVB polypeptides, or one or more nucleic acids comprising nucleotide sequences encoding the one or more CVB polypeptides,
in a volume of 500 μL.

10. The composition of claim 9, wherein the composition comprises:
a) CVB1 in an amount of from 2.0×10⁷ IVP to 5.0×10⁷ IVP;
b) CVB2 in an amount of from 1.0×10⁷ IVP to 3.0×10⁷ IVP;
c) CVB3 in an amount of from 1.0×10⁸ IVP to 3.0×10⁸ IVP;
d) CVB4 in an amount of from 2.0×10⁷ IVP to 5.0×10⁷ IVP; and
e) CVB5 in an amount of from 4.0×10⁷ IVP to 8.0×10⁷ IVP,
or an equivalent amount of VLP, one or more CVB polypeptides, or one or more nucleic acids comprising nucleotide sequences encoding the one or more CVB polypeptides; or
wherein the composition comprises:
a) CVB1 in an amount of from 3.0×10⁷ IVP to 4.0×10⁷ IVP;
b) CVB2 in an amount of from 1.0×10⁷ IVP to 2.0×10⁷ IVP;
c) CVB3 in an amount of from 1.0×10⁸ IVP to 2.0×10⁸ IVP;
d) CVB4 in an amount of from 3.0×10⁷ IVP to 4.0×10⁷ IVP; and
e) CVB5 in an amount of from 6.0×10⁷ IVP to 6.0×10⁷ IVP,
or an equivalent amount of VLP, one or more CVB polypeptides, or one or more nucleic acids comprising nucleotide sequences encoding the one or more CVB polypeptides; or
wherein the composition comprises:
a) CVB1 in an amount of 3.3×10⁷ IVP;
b) CVB2 in an amount of 1.1×10⁷ IVP;
c) CVB3 in an amount of 1.5×10⁸ IVP;
d) CVB4 in an amount of 3.2×10⁷ IVP; and
e) CVB5 in an amount of 6.3×10⁷ IVP,
or an equivalent amount of VLP, one or more CVB polypeptides, or one or more nucleic acids comprising nucleotide sequences encoding the one or more CVB polypeptides,
in a volume of 100 μl.

11.-12. (canceled)

13. The composition of claim 9, wherein the composition comprises:
a) CVB1 in an amount of 1.6×10⁸ IVP;
b) CVB2 in an amount of 5.5×10⁷ IVP;
c) CVB3 in an amount of 7.5×10⁸ IVP;
d) CVB4 in an amount of 1.6×10⁸ IVP; and
e) CVB5 in an amount of 3.1×10⁸ IVP,
or an equivalent amount of VLP, one or more CVB polypeptides, or one or more nucleic acids comprising nucleotide sequences encoding the one or more CVB polypeptides,
in a volume of 500 μL.

14. The composition of claim 9, wherein the composition comprises:
a) CVB1 protein in an amount of 2.8 μg;
b) CVB2 protein in an amount of 1.7 μg;
c) CVB3 protein in an amount of 2.7 μg;
d) CVB4 protein in an amount of 1.2 μg; and
e) CVB5 protein in an amount of 2.3 μg,
in a volume of 100 μl; or
wherein the composition comprises:
a) CVB1 protein in an amount of 14 μg;
b) CVB2 protein in an amount of 8.5 μg;
c) CVB3 protein in an amount of 13.5 μg;
d) CVB4 protein in an amount of 6.0 μg; and
e) CVB5 protein in an amount of 11.5 μg,
in a volume of 500 μl.

15. (canceled)

16. The composition of claim 1, wherein the IVP are formalin inactivated.

17. The composition of claim 1, wherein the composition comprises an adjuvant and/or saline.

18. (canceled)

19. A method of inducing an immune response to coxsackievirus B (CVB) in an individual, the method comprising administering to the individual a composition of claim 1.

20. The method of claim 19, wherein the composition is administered intramuscularly or subcutaneously.

21. (canceled)

22. A method of reducing the likelihood that an individual will develop an acute coxsackievirus B (CVB) infection or a CVB infection-associated disease, the method comprising administering to the individual a composition of claim 1.

23. The method of claim 19, wherein the individual is a neonate or a pregnant woman.

24. (canceled)

25. The method of claim 19, wherein the individual is at increased risk of developing type 1 diabetes.

26. The method of claim 19, wherein the individual is at increased risk of developing celiac disease.

27. The method of claim 19, wherein the individual is a carrier of an HLA DR3 and/or an HLA DR4 allele.

28. The method of claim 19, wherein the individual was seropositive for viral neutralizing titer to one or more of CVB1, CVB2, CVB3, CVB4, and CVB5 prior to said administering.

29. The method of claim 19, wherein the individual was seronegative for viral neutralizing titer to one or more of CVB1, CVB2, CVB3, CVB4, and CVB5 prior to said administering.


SEQUENCE LISTING (1 sequence)

SEQ ID NO: 1 - protein, 2183 aa, Coxsackie B virus type 4
MGAQVSTQKTGAHETSLNASGSSIIHYTNINYYKDAASNSANRQDFTQDPSKFTEPVKDVMIKTLPALNSPTVEECGYSDRVRSITLGNSTITTQECANVVVGYGVWPDYLSDEEATAEDQPTQPDVATCRFYTLNSVKWEMQSAGWWWKFPDALSEMGLFGQNMQYHYLGRSGYTIHVQCNASKFHQGCLLVVCVPEAEMGCTNVENAPTYGDLCGGETAKQFEQNAVTGETAVQTAVCNAGMGVGVGNLTIYPHQWINLRTNNSATIVMPYINSVPMDNMFRHNNFTLMIIPFAPLDYVTGASSYIPITVTVAPMSAEYNGLRLAGHQGLPTMLTPGSTQFLTSDDFQSPSAMPQFDVTPEMNIPGQVRNLMEIAEVDSVVPLNNLKANIKTMEAYRVQVRSTDEMGGQIFGFPLQPGASSVLQRTLLGEILNYYTHWSGSLKLTFVFCGSAMATGKFLLAYSPPGAGAPDSRKNAMLGTHVIWDVGLQSSCVLCVPWISQTHYRFVVDDKYTASGFISCWYQTNVIVPAEAQKSCYIMCFVSACNDFSVRMLRDTQFIKQDNFYQGPTEDSVERAMGRVADTIARGPSNSEQIPALTAVETGHTSQVDPSDTMQTRHVHNYHSRSESSIENFLCRSACVIYIKYSSAESNNLKRYAEWVINTRQVAQLRRKMEMFTYVRCDMELTFVITSHQEMSTATNSDVPVQTHQIMYVPPGGPVPTSVDDYVWQTSTNPSIFWTEGNAPPRMSIPFMSIGNAYTMFYDGWSNFSRDGIYGYNSLNNMGTIYARHVNDSSPGGLTSTIRIYFKPKHVKAYVPRPPRLCQYKKAKNVNFEVEAVTTERASLVTTGPHGQQSGAVYVGNYRIINRHLATNFDWQNCVWEDYNRDLLVSTTTAHGCDTIARCQCTTGVYFCASKNKHYPVAFEGPGLVEVQESEYYPKRYQSHVLLAAGFSEPGDCGGILRCEHGVIGIVTMGGEGIVGFADVRDLLWLEDDAMEQGVKDYVEQLGNAFGSGFTNQICEQVNLLKESLVGQDSILEKSLKALVKIISALVIVVRNHDDLITVTATLALIGCTSSPWRWLKHKVSQYYGIPMAERQNNNWLKKFTEMTNACKGMEWIAVKIQKFIEWLKVKILPEVREKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKDKGWLVPETTSTLQIEKHVSRAFICLQALTTFVSVAGIIYIIYKLFAGFQGAYTGLPNQKPRVPTLRQAKVQGPAFEFAVAMMKRNASTVKTEYGEFTMLGIYDRWAVLPRHAKPGPTILMNDQEVGVLDAKELVDKDGTNLELTLLKLNRNEKFRDIRGFLAREEVEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIENSKDAGFPIINTPSKTKLEPSVFHQVFEGNKEPAVLRNGDPRLKANFEEAIFSKYIGNVNTHVDEYMQEAVDHYAGQLATLDISTEPMKLEDAVYGTEGLEALDLTTSAGYPYVALGIKKRDILSKKTRDLTKLKECMDKYGLNLPMVTYVKDELRSAEKVAKGKSRLIEASSLNDSVAMRQTFGNLYKTFHLNPGIVTGSAVGCDPDVFWSKIPVMLDGHLIAFDYSGYDASLSPVWFACLKLLLEKLGYTNKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEHEYEEFIRKIRSVPVGRCLTLPAFSTLRRKWLDSF